(19) United States
(12) Patent Application Publication
(10) Pub. No.: US 2021/0347869 A1
(43) Pub. Date: Nov. 11, 2021

(54) METHODS AND COMPOSITIONS FOR TREATING TAUOPATHIES

(71) Applicant: THE UNIVERSITY OF QUEENSLAND, St. Lucia, Queensland (AU)

(72) Inventors: Juergen GOETZ, St Lucia, Queensland (AU); Rebecca NISBET, St Lucia, Queensland (AU); Phillip JANOWICZ, St Lucia, Queensland (AU); Gerhard LEINENGA, St Lucia, Queensland (AU)

(73) Assignee: THE UNIVERSITY OF QUEENSLAND, St. Lucia, Queensland (AU)

(21) Appl. No.: 17/285,303

(22) PCT Filed: Oct. 17, 2018
(86) PCT No.: PCT/AU2018/051129

§ 371 Date: Apr. 14, 2021

(51) Int. Cl. C07K 16/18; A61K 41/00

(52) U.S. Cl. CPC C07K 16/18; A61K 41/0047; C07K 2317/56; C07K 2317/565; C07K 2317/567; C07K 2317/55; C07K 2317/624; C07K 2317/626; C07K 2317/62; C07K 2317/54; C07K 2317/622

(57) ABSTRACT

The invention relates to tau, to antibodies and related fragments thereof for binding to tau, to production of said antibodies and fragments and to use of said antibodies and fragments for detection and therapy of various conditions, including tauopathies. The invention also relates to compositions comprising tau antibodies for binding to tau and their use in combination with acoustic energy.

### FIELD OF THE INVENTION

The invention relates to tau, to antibodies and related fragments thereof for binding to tau, to production of said antibodies and fragments and to use of said antibodies and fragments for detection and therapy of various conditions, including tauopathies.

### BACKGROUND OF THE INVENTION

Alzheimer's disease (AD) and related tauopathies are progressive neurodegenerative diseases for which there is no cure. AD is characterized by the extracellular deposition of amyloid-beta (Aβ) as amyloid plaques and the intracellular deposition of tau as neurofibrillary tangles, with the latter directly correlating with dementia in AD patients. Reducing tau levels abrogates the toxic effects of pathological tau, but also reduces Aβ-mediated toxicity, making tau an attractive therapeutic target.

The bloodbrain barrier (BBB) limits the passage of molecules from the blood into the central nervous system and remains a significant obstacle for neurological therapeutics, particularly for molecules greater than 800 Da such as antibodies. This challenges the therapeutic potential of antibody-based treatments of neurodegenerative diseases and may, at least partially, account for the low clinical success rate of several anti-Aβ therapies. Furthermore, anti-tau therapeutics present an additional challenge because they must also cross the neuronal cell membrane to interact with the majority of tau that accumulates intracellularly.

There exists a need for new and/or improved approaches to treat diseases associated with tau. There also exists a need for more efficient methods of tau antibody delivery and/or neuronal uptake.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the invention provides for a method of delivering an antigen binding site that binds to or specifically binds to tau in a subject comprising:

administering to the subject an antigen binding site that binds to or specifically binds to tau, and

administering acoustic energy to a region of the brain of the subject;

wherein the application of acoustic energy acts as a means to permit or facilitate the antigen binding site to pass through the blood-brain barrier (BBB) of the subject,

thereby delivering the antigen binding that binds to or specifically binds to tau.

In any aspect of the present invention, the antigen binding site has a molecular weight greater than about 29 kDa. Preferably, the antigen binding site has a molecular weight greater than an scFv. More preferably, the antigen binding site has a molecular weight of between about 29 and about 156 kDa.

In any aspect of the present invention, the antigen binding site comprises a fragment crystallizable region (Fc region).

In any aspect of the present invention, the antigen binding site is an IgG type. Preferably, the IgG isotype is IgG1 or IgG2. Preferably, the IgG2 isotype is IgG2a.

Preferably, the antigen binding site of the invention binds to or specifically binds to human tau. In one embodiment, the human tau comprises, consists essentially of or consists of the amino acid sequence shown in SEQ ID NO: 33. Preferably, the antigen binding site binds with higher affinity to 2N isoform of the tau protein than any other tau isoform, particularly 1N or 0N.

Preferably, the antigen binding site binds to or specifically binds to a human tau molecule comprising, consisting essentially of or consisting of an amino acid sequence of residues, or residues equivalent to, 84 to 97 of the human tau isoform, tau441. In one embodiment, the amino acid sequence of residues 84 to 97 of tau441 is shown in SEQ ID NO: 34.

In any aspect of the present invention, the antigen binding site binds to a peptide comprising, consisting essentially of or consisting of the sequence: TEIPEGITAEEAGI (SEQ ID NO:34).

In any aspect of the present invention, the antigen binding site is not an scFv.

In any aspect of the present invention, the antigen binding site is any antigen binding site of the invention as described herein.

In any aspect of the present invention, the tau may be intracellular or extracellular. In any aspect, the tau may be in a glial cell or a neuron. Preferably, the intracellular tau is in a neuron. Preferably, the neuron is in the brain. Therefore, in any method of the invention, the administration or application of acoustic energy may permit or facilitate the antigen binding site to pass through the BBB and/or through the cell membrane thereby enabling the antigen binding site to interact with extracellular or intracellular tau, respectively.

In any aspect of the present invention, the application of acoustic energy acts as a means to permit or facilitate the antigen binding site to pass through the blood-brain barrier (BBB) of the subject

In any aspect, the tau may be any pathological form. For example, the tau may be filaments such as paired helical filaments (PHF) or aggregates such as neurofibrillary tangles (NFT).

In any aspect of present invention, the antigen binding site has a dissociation constant (K_{D}) of less than 460 nM, less that 410 nM, less than 400 nM, less than 390 nM, or less than 380 nM.

In any aspect of the present invention, the acoustic energy is ultrasound. The ultrasound may be scanning ultrasound (SUS) or non-scanning ultrasound. In an embodiment, the SUS or non-scanning ultrasound is administered with microbubbles to disrupt the blood-brain barrier. The administration of microbubbles may be before, after or during the administration of SUS or non-scanning ultrasound. In some instances, the antibody or antigen-binding fragment is administered to the subject before, at the same time and/or after the subject has received scanning ultrasound (SUS) or non-scanning ultrasound.

In another aspect the present invention provides a method of improving cognitive function in a subject, the method comprising, consisting essentially of or consisting of the steps of:

administering to the subject an antigen binding site that binds to or specifically binds to tau;

identifying a region of the brain of the subject to which acoustic energy is to be applied; and

applying a clinically safe level of acoustic energy to the region, thereby saturating or substantially saturating the region with acoustic energy;

thereby improving cognitive function in the subject.

In any aspect of the present invention, the subject may have impaired cognitive function. Impaired cognitive function may be determined by any method as described herein. Further, in any method or use of the invention, the subject may be identified as having impaired cognitive function. In any method of the invention, the method further comprises a step of identifying an individual with impaired cognitive function.

In another aspect the present invention provides a method of improving cognitive function in a subject with a condition associated with a pathological form of tau, the method comprising, consisting essentially of or consisting of the steps of:

administering to a subject an antigen binding site that binds to or specifically binds to tau;

identifying a region of the brain of the subject to which acoustic energy is to be applied; and

applying a clinically safe level of acoustic energy to the region, thereby saturating or substantially saturating the region with acoustic energy;

thereby improving cognitive function in the subject.

In any aspect of the invention, the condition or disease for treatment is one associated with or caused by a pathological form of tau. Preferably, the tau is in the form of an oligomer, aggregate or deposit. The condition or disease is a tauopathy. The tauopathy may be any one described herein.

In another aspect the present invention provides a method of improving memory, motor skills and/or executive functions in a subject with impaired memory function, the method including the steps of:

administering to a subject an antigen binding site that binds to or specifically binds to tau;

identifying a region of the brain of the subject to which acoustic energy is to be applied; and

applying a clinically safe level of acoustic energy to the region, thereby saturating or substantially saturating the region with acoustic energy;

thereby improving memory, motor skills and/or executive functions in the subject.

The present invention provides a method of improving memory, motor skills, executive functions and/or cognitive function in a subject with impaired memory and/or cognitive function, the method including the steps of:

providing a subject with impaired memory, motor skills, executive functions, and/or cognitive function;

administering to the subject an antigen binding site that binds to or specifically binds to tau;

identifying a region of the brain of the subject to which acoustic energy is to be applied; and

applying a clinically safe level of acoustic energy to the region, thereby saturating or substantially saturating the region with acoustic energy;

thereby improving memory, motor skills, executive functions and/or cognitive function in the subject.

Preferably, identifying a region of the brain as described herein includes determining a volume of the brain on the basis of symptoms displayed by the subject, typically clinically observable or biochemically detectable symptoms, or determining a volume of the brain on the basis of a known association with a tauopathy, in particular those associated with protein oligomers, aggregates or deposits, or determining a volume of the brain including a volume surrounding an site having intracellular and or extracellular tau protein in a pathogenic form, such as oligomers, an aggregate or deposit.

The method of the invention further includes determining a plurality of discrete application sites for application of acoustic energy to saturate or substantially saturate the region with acoustic energy.

The method further includes determining a scanning path along which acoustic energy is to be applied to saturate or substantially saturate the region with acoustic energy. Preferably, the method further includes determining a plurality of discrete application sites for application of acoustic energy along the scanning path.

Typically, applying a clinically safe level of acoustic energy to the region includes providing acoustic energy continuously, or at application sites, along a scanning path.

In one embodiment, the scanning path is defined by a pre-determined pattern.

The scanning path may be selected from the group consisting of linear, serpentine, a raster pattern, spiral and random.

Each application site may be spaced along the scanning path or each subsequent application site may overlap with the previous application site.

Applying a clinically safe level of acoustic energy to the region, includes applying acoustic energy at an application site such that a corresponding treatment volume is therapeutically affected by acoustic energy, and wherein saturating or substantially saturating the region with acoustic energy includes applying acoustic energy at a plurality of discrete application sites or one or more extended application sites such that the corresponding treatment volume(s) correspond substantially with the region.

The plurality of application sites may be selected such that treatment volumes of at least some sites overlap to form a group of treatment volumes that corresponds substantially with the region.

The plurality of application sites may be selected such that their corresponding treatment volumes overlap to form a contiguous treatment volume that corresponds substantially with the region.

A region of the brain may the entire brain, hemisphere, forebrain or a region of the brain of the subject known to be associated with a condition involving the presence of proteins adopting pathogenic structures in an extracellular region. Such structures may be oligomers, aggregates and/or deposits. The region may be any one or more of the following cerebrum, cerebral hemisphere, telencephalon, forebrain, cortex, frontal lobe, prefrontal cortex, precentral gyrus, primary motor cortex, premotor cortex, temporal lobe, auditory cortex, inferior temporal cortex, superior temporal gyrus, fusiform gyrus, parahippocampal gyrus, entorhinal cortex, parietal lobe, somatosensory cortex, postcentral gyrus, occipital lobe, visual cortex, insular cortex, cingulate cortex, subcortical, hippocampus, dentate gyrus, cornu ammonis, amygdala, basal ganglia, striatum, caudate, putamen, nucleus accumbens, olfactory tubercle, globus pallidus, subthalamic nuclei, piriform cortex, olfactory bulb, fornix, mammillary bodies, basal forebrain, nucleus basalis Meynert, diencephalon, thalamus, hypothalamus, midbrain, tectum, tegmentum, substantia nigra, hindbrain, myelencephalon, medulla oblongata, metencephalon, pons, cerebellum, spinal cord, brain stem and cranial nerves.

In a subject identified as having Alzheimer's disease the region may be selected from the group consisting of cerebrum, cerebral hemisphere, telencephalon, forebrain, cortex, frontal lobe, prefrontal cortex, precentral gyrus, temporal lobe, auditory cortex, inferior temporal cortex, superior temporal gyrus, fusiform gyrus, parahippocampal gyrus, entorhinal cortex, insular cortex, cingulate cortex, subcortical, hippocampus, dentate gyrus, cornu ammonis, amygdala, piriform cortex, olfactory bulb, fornix, mammillary bodies, basal forebrain and nucleus basalis of Meynert.

In any embodiment of the invention, the region is not solely identified as a plaque. The region may be an aggregate or deposit of pathological protein.

As used herein the acoustic energy may provide conditions for an increase in the permeability of the blood-brain barrier, or activating microglia. Conditions for an increase in the permeability of the blood-brain barrier are described further herein.

Preferably, a clinically safe level of acoustic energy does not result in detectable heating, brain swelling, red blood cell extravasation, haemorrhage or edema.

Acoustic energy used in the invention may be scanning ultrasound (SUS). Ultrasound may be focussed or unfocussed.

A subject with impaired cognitive function, motor skills, executive functions and/or memory function may be identified as having a neurodegenerative disease associated with, or caused by, the presence, over-expression or accumulation of tau. As used herein, pathological tau refers to a form or an amount of tau that is not present in a normal individual, i.e. one without impaired cognitive function, motor skills, executive functions and/or memory function.

Typically, an improvement in cognitive function or memory is determined by standardised neuropsychological testing.

In any aspect of the present invention, the administration of the antigen binding site and the application of acoustic energy may be sequential or concurrent. Alternatively, administration and application may be done at different times. In an embodiment, the SUS is administered to the entire or whole brain as a means to allow for an antigen binding site to pass through the blood-brain barrier.

In any aspect of the present invention, the antigen binding site with or without SUS inhibits or prevents the accumulation, or deposition of tau aggregates intracellularly or extracellularly in the central nervous system. Preferably, the administration of the antigen binding site with or without SUS improves cognitive function in a subject with a tauopathy.

Preferably, the antigen binding site of the invention binds to or specifically binds to human tau. Preferably, the antigen binding site binds to or specifically binds to a human tau molecule comprising, consisting essentially of or consisting of an amino acid sequence as shown in SEQ ID NO: 33. Preferably, the antigen binding site is specific for 2N isoform of the tau protein. In one embodiment, the antibody binds to a peptide comprising, consisting essentially of or consisting of the sequence: TEIPEGITAEEAGI (SEQ ID NO:34) or a fragment thereof.

In another aspect the present invention provides a method for delivery of an antigen binding site that binds to or specifically binds to tau, the method comprising:

administering an antigen binding site that binds to or specifically binds to a peptide comprising, consisting essentially of or consisting of an amino acid sequence as shown in SEQ ID NO: 34; and

administering acoustic energy to the brain of a subject,

wherein the application of acoustic energy acts as a means to permit or facilitate the antigen binding site to pass through the blood-brain barrier (BBB), thereby delivering the antigen binding site.

In another aspect the invention provides an antigen binding site for binding to tau, the antigen binding site comprising:

FR1-CDR1-FR2-CDR2-FR3-CDR3-FR4-linker-FR1a-CDR1a-FR2a-CDR2a-FR3a-CDR3a-FR4a

wherein:

FR1, FR2, FR3 and FR4 are each framework regions;

CDR1, CDR2 and CDR3 are each complementarity determining regions;

FR1a, FR2a, FR3a and FR4a are each framework regions;

CDR1a, CDR2a and CDR3a are each complementarity determining regions;

wherein the sequence of any of the framework regions or complementarity determining regions are as described herein.

In one aspect, the invention provides an antigen binding site for binding to tau, the antigen binding site including:

FR1-CDR1-FR2-CDR2-FR3-CDR3-FR4-linker-FR1a-CDR1a-FR2a-CDR2a-FR3a-CDR3a-FR4a

wherein:

FR1, FR2, FR3 and FR4 are each framework regions;

CDR1, CDR2 and CDR3 are each complementarity determining regions;

FR1a, FR2a, FR3a and FR4a are each framework regions;

CDR1a, CDR2a and CDR3a are each complementarity determining regions;

wherein the sequence of any of the complementarity determining regions have an amino acid sequence as described in Table 1 below. Preferably, the framework regions have an amino acid sequence also as described in Table 1 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

In one aspect the present invention also provides an antigen binding site comprising an antigen binding domain of an antibody, wherein the antigen binding domain binds to or specifically binds to tau, wherein the antigen binding domain comprises at least one of:

(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO:4 or 38, a CDR2 comprising a sequence at least about 80%, at least 85%, at least 90%, at least 92%, at least 95%, at least 97%, at least 99% identical to a sequence set in SEQ ID NO:5 or 39 and a CDR3 comprising a sequence at least about 80%, at least 85%, at least 90%, at least 92%, at least 95%, at least 97%, at least 99% identical to a sequence set forth in SEQ ID NO: 6 or 40;

(ii) a VH comprising a sequence at least about 95% or 96% or 97% or 98% or 99% identical to a sequence set forth in SEQ ID NO: 8 or 42;

(iii) a VL comprising a CDR1 comprising a sequence at least about 80%, at least 85%, at least 90%, at least 92%, at least 95%, at least 97%, at least 99% identical to a sequence set forth in SEQ ID NO: 1 or 35, a CDR2 comprising a sequence at least about 80%, at least 85%, at least 90%, at least 92%, at least 95%, at least 97%, at least 99% identical to a sequence set forth in SEQ ID NO: 2 or 36 and a CDR3 comprising a sequence at least about 80%, at least 85%, at least 90%, at least 92%, at least 95%, at least 97%, at least 99% identical to a sequence set forth in SEQ ID NO: 3 or 37;

(iv) a VL comprising a sequence at least about 95% identical to a sequence set forth in SEQ ID NO: 7 or 41;

(v) a VH comprising a CDR1 comprising a sequence set forth in SEQ ID NO: 4 or 38, a CDR2 comprising a sequence set forth between in SEQ ID NO: 5 or 39 and a CDR3 comprising a sequence set forth in SEQ ID NO: 6 or 40;

(vi) a VH comprising a sequence set forth in SEQ ID NO: 8 or 42;

(vii) a VL comprising a CDR1 comprising a sequence set SEQ ID NO: 1 or 35, a CDR2 comprising a sequence set forth in SEQ ID NO: 2 or 36 and a CDR3 comprising a sequence set forth in SEQ ID NO: 3 or 37;

(viii) a VL comprising a sequence set forth in SEQ ID NO: 7 or 41;

(ix) a VH comprising a CDR1 comprising a sequence set forth in SEQ ID NO: 4 or 38, a CDR2 comprising a sequence set forth between in SEQ ID NO: 5 or 39 and a CDR3 comprising a sequence set forth in SEQ ID NO: 6 or 40; and a VL comprising a CDR1 comprising a sequence set SEQ ID NO: 1 or 35, a CDR2 comprising a sequence set forth in SEQ ID NO: 2 or 36 and a CDR3 comprising a sequence set forth in SEQ ID NO: 3 or 37; or

(x) a VH comprising a sequence set forth in SEQ ID NO: 8 or 42 and a VL comprising a sequence set forth in SEQ ID NO: 7 or 41.

In any aspect of the invention, the antigen binding domain further comprises at least one of:

(i) a VH comprising a framework region (FR) 1 comprising a sequence at least about 80%, at least 85%, at least 90%, at least 92%, at least 95%, at least 97%, at least 99% identical to a sequence set forth in SEQ ID NO:21 or 55, a FR2 comprising a sequence at least about 80%, at least 85%, at least 90%, at least 92%, at least 95%, at least 97%, at least 99% identical to a sequence set in SEQ ID NO:22 or 56, a FR3 comprising a sequence at least about 80%, at least 85%, at least 90%, at least 92%, at least 95%, at least 97%, at least 99% identical to a sequence set forth in SEQ ID NO: 23 or 57, and a FR4 comprising a sequence at least about 80%, at least 85%, at least 90%, at least 92%, at least 95%, at least 97%, at least 99% identical to a sequence set forth in SEQ ID NO: 24 or 58;

(ii) a VL comprising a FR1 comprising a sequence at least about 80%, at least 85%, at least 90%, at least 92%, at least 95%, at least 97%, at least 99% identical to a sequence set forth in SEQ ID NO: 17 or 51, a FR2 comprising a sequence at least about 80%, at least 85%, at least 90%, at least 92%, at least 95%, at least 97%, at least 99% identical to a sequence set forth in SEQ ID NO: 18 or 52, a FR3 comprising a sequence at least about 80%, at least 85%, at least 90%, at least 92%, at least 95%, at least 97%, at least 99% identical to a sequence set forth in SEQ ID NO: 19 or 53, and a FR4 comprising a sequence at least about 80%, at least 85%, at least 90%, at least 92%, at least 95%, at least 97%, at least 99% identical to a sequence set forth in SEQ ID NO: 20 or 54;

(iii) a VH comprising a FR1 comprising a sequence set forth in SEQ ID NO: 21 or 55, a FR2 comprising a sequence set forth between in SEQ ID NO: 22 or 56, a FR3 comprising a sequence set forth in SEQ ID NO: 23 or 57, and a FR4 comprising a sequence set forth in SEQ ID NO: 24 or 58;

(iv) a VL comprising a FR1 comprising a sequence set forth in SEQ ID NO: 17 or 51, a FR2 comprising a sequence set forth between in SEQ ID NO: 18 or 52, a FR3 comprising a sequence set forth in SEQ ID NO: 19 or 53, and a FR4 comprising a sequence set forth in SEQ ID NO: 20 or 54; or

(v) a VH comprising a FR1 comprising a sequence set forth in SEQ ID NO: 21 or 55, a FR2 comprising a sequence set forth between in SEQ ID NO: 22 or 56, a FR3 comprising a sequence set forth in SEQ ID NO: 23 or 57, and a FR4 comprising a sequence set forth in SEQ ID NO: 24 or 58; and a VL comprising a FR1 comprising a sequence set forth in SEQ ID NO: 17 or 51, a FR2 comprising a sequence set forth between in SEQ ID NO: 18 or 52, a FR3 comprising a sequence set forth in SEQ ID NO: 19 or 53, and a FR4 comprising a sequence set forth in SEQ ID NO: 20 or 54.

As described herein, the antigen binding site may be in the form of:

(i) a single chain Fv fragment (scFv);

(ii) a dimeric scFv (di-scFv);

(iii) one of (i) or (ii) linked to a constant region of an antibody, Fc or a heavy chain constant domain (CH) 2 and/or CH3; or

(iv) one of (i) or (ii) linked to a protein that binds to tau.

Further, as described herein, the antigen binding site may be in the form of:

(i) a diabody;

(ii) a triabody;

(iii) a tetrabody;

(iv) a Fab;

(v) a F(ab′)2;

(vi) a Fv;

(vii) one of (i) to (vi) linked to a constant region of an antibody, Fc or a heavy chain constant domain (CH) 2 and/or CH3; or

(viii) one of (i) to (vi) linked to a protein that binds to tau.

Further, as described herein, the antigen binding site may be in the form of:
(i) IgG1;
(ii) IgG2a, IgG2b, IgG3;
(iii) one of (i) to (ii) linked to a constant region of an antibody, Fc or a heavy chain constant domain (CH) 2 and/or CH3;
(iv) one of (i) to (vi) linked to a protein that binds to tau.

The foregoing antigen binding sites can also be referred to as antigen binding domains of antibodies.

Preferably, an antigen binding site as described herein is an antibody or antigen binding fragment thereof. Typically, the antigen binding site is an antibody, for example, a monoclonal antibody.

As used herein the antigen binding site may be a variable domain.

In any embodiment of the invention, the antigen binding site may be a synthetic binding site. For example, the binding site may be chimeric, humanized, human, synhumanized, primatized, de-immunized or a composite antigen binding site.

The present invention also provides a tau antibody comprising a light chain variable region and a heavy chain variable region,

wherein said light chain variable region comprises:

a LCDR1 as set forth in SEQ ID NO:1 or 35, a LCDR2 as set forth in SEQ ID NO:2 or 36 and a LCDR3 as set forth in SEQ ID NO:3 or 37; and

wherein said heavy chain variable region comprises:

a HCDR1 as set forth in SEQ ID NO:4 or 38, a HCDR2 as set forth in SEQ ID NO:5 or 39, and a HCDR3 as set forth in SEQ ID NO:6 or 40.

In any aspect of the invention, a tau antibody comprises a light chain variable region that comprises the sequence of SEQ ID NO:7 or 41.

In any aspect of the invention, a tau antibody comprises a heavy chain variable region that comprises the sequence of SEQ ID NO:8 or 42.

In any aspect of the invention, a tau antibody comprises a light chain variable region that comprises a FR L1 as set forth in SEQ ID NO:17 or 51, FR L2 as set forth in SEQ ID NO:18 or 52, a FR L3 as set forth in SEQ ID NO:19 or 53 and a FR L4 as set forth in SEQ ID NO:20 or 54.

In any aspect of the invention, a tau antibody comprises a heavy chain variable region that comprises a FR H1 as set forth in SEQ ID NO:21 or 55, FR H2 as set forth in SEQ ID NO:22 or 56, a FR H3 as set forth in SEQ ID NO:23 or 57 and a FR H4 as set forth in SEQ ID NO:24 or 58.

In any aspect or embodiment, the antibody is a naked antibody. Specifically, the antibody is in a non-conjugated form and is not adapted to form a conjugate.

As used herein, the complementarity determining region sequences (CDRs) of an antigen binding site of the invention are defined according to the IMGT or the Chothia numbering system.

Reference herein to a protein or antibody that “binds to” tau provides literal support for a protein or antibody that “binds specifically to” or “specifically binds to” tau.

The present invention also provides antigen binding domains or antigen binding fragments of the foregoing antibodies.

An antigen binding site of the present invention as described herein may be used in any method, use or composition of the invention as described herein.

The invention also provides a fusion protein comprising an antigen binding site, immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody as described herein.

The invention also provides a conjugate in the form of an antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody or fusion protein as described herein conjugated to a label or a cytotoxic agent.

The invention also provides an antibody for binding to an antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein, or conjugate as described herein.

The invention also provides a nucleic acid encoding an antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein.

In one example, such a nucleic acid is included in an expression construct in which the nucleic acid is operably linked to a promoter. Such an expression construct can be in a vector, e.g., a plasmid.

In examples of the invention directed to single polypeptide chain antigen binding sites, the expression construct may comprise a promoter linked to a nucleic acid encoding that polypeptide chain.

In examples directed to multiple polypeptide chains that form an antigen binding site, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:

(i) a promoter

(ii) a nucleic acid encoding a first polypeptide;

(iii) an internal ribosome entry site; and

(iv) a nucleic acid encoding a second polypeptide, wherein the first polypeptide comprises a VH and the second polypeptide comprises a VL, or vice versa.

The present invention also contemplates separate expression constructs one of which encodes a first polypeptide comprising a VH and another of which encodes a second polypeptide comprising a VL. For example, the present invention also provides a composition comprising:

(i) a first expression construct comprising a nucleic acid encoding a polypeptide comprising a VH operably linked to a promoter; and

(ii) a second expression construct comprising a nucleic acid encoding a polypeptide comprising a VL operably linked to a promoter.

The invention provides a cell comprising a vector or nucleic acid described herein. Preferably, the cell is isolated, substantially purified or recombinant. In one example, the cell comprises the expression construct of the invention or:

(i) a first expression construct comprising a nucleic acid encoding a polypeptide comprising a VH operably linked to a promoter; and

(ii) a second expression construct comprising a nucleic acid encoding a polypeptide comprising a VL operably linked to a promoter,

wherein the first and second polypeptides associate to form an antigen binding site of the present invention.

Examples of cells of the present invention include bacterial cells, yeast cells, insect cells or mammalian cells.

The invention also provides a pharmaceutical composition comprising an antigen binding site, or comprising a CDR and/or FR sequence as described herein, or an immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody, fusion protein, or conjugate as described herein and a pharmaceutically acceptable carrier, diluent or excipient.

The invention also provides a diagnostic composition comprising an antigen binding site, or comprising a CDR and/or FR sequence as described herein, or antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein, a diluent and optionally a label.

The invention also provides a kit or article of manufacture comprising an antigen binding site, or comprising a CDR and/or FR sequence as described herein or an immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein.

An antigen binding site, a protein or antibody as described herein may comprise a human constant region, e.g., an IgG constant region, such as an IgG1, IgG2, IgG3 or IgG4 constant region or mixtures thereof. In the case of an antibody or protein comprising a VH and a VL, the VH can be linked to a heavy chain constant region and the VL can be linked to a light chain constant region.

The functional characteristics of an antigen binding site of the invention will be taken to apply mutatis mutandis to an antibody of the invention.

An antigen binding site as described herein may be purified, substantially purified, isolated and/or recombinant.

An antigen binding site of the invention may be part of a supernatant taken from media in which a hybridoma expressing an antigen binding site of the invention has been grown.

In another aspect, the present invention also provides a method for inhibiting or preventing the accumulation or deposition of pathological tau protein aggregates in the central nervous system in a subject, comprising administering to the subject an effective amount of an antigen binding site of the invention, thereby inhibiting or preventing the accumulation or deposition of pathological tau protein aggregates in the central nervous system in a subject.

In another aspect, the invention provides a method for treating, delaying, reducing, inhibiting or preventing the accumulation or deposition of pathological protein aggregates in the central nervous system in a subject, comprising

administering an antigen binding site of the invention as described herein; and

administering acoustic energy to the brain of a subject,

wherein the application of acoustic energy acts as a means to permit or facilitate the antigen binding site to pass through the blood-brain barrier (BBB),

treating, delaying, reducing, inhibiting or preventing the accumulation or deposition of pathological protein aggregates in the central nervous system in a subject.

In another aspect, the invention provides a method for treating, delaying, inhibiting or preventing the progression of a tauopathy comprising administering an antigen binding site of the present invention as described herein to a subject in need thereof, thereby treating, delaying, inhibiting or preventing the progression of a tauopathy in a subject in need thereof. Preferably, the method further comprises applying acoustic energy to the subject in need thereof, preferably scanning ultrasound (SUS).

The invention provides a use of an antigen binding site of the present invention as described herein, in the preparation of a medicament for method for treating, inhibiting, delaying or reducing the progression of a tauopathy in a subject in need thereof. In another aspect, the use is suitable for applying acoustic energy to the subject in need thereof, preferably scanning ultrasound (SUS).

Typically, a method of the invention also includes the step of administering an agent to promote the increase in permeability of the blood-brain barrier. In a preferred form that agent promotes cavitation. An agent that promotes cavitation may be a microbubble agent as described herein.

In any embodiment of the invention, the method may further comprise a step of administering a microbubble agent to the subject. Administration of microbubbles may be before, after or during the administration of SUS.

The microbubble may be provided to the subject by continuous infusion or a single bolus. The infusion may occur sequentially to, or following the start of, or simultaneously with, the application of the ultrasound.

In an embodiment, the step of applying the acoustic energy is repeated.

Any method of the invention described herein may also further include the step of determining that the permeability of the blood-brain barrier has increased.

The acoustic energy may be applied in a method of the invention at a pressure greater than 0.4 MPa. Typically this pressure is used when application of the acoustic energy is outside the skull, i.e. transcranially. Otherwise, the acoustic energy may be applied with a mechanical index of between 0.1 and 2.

In some embodiments of the methods of the invention, the tauopathy is Alzheimer's disease, Amyotrophic lateral sclerosis/parkinsonism-dementia complex, Argyrophilic grain dementia, Corticobasal degeneration, Creutzfeldt-Jakob disease, Dementia pugilistica, Diffuse neurofibrillary tangles with calcification, Down's syndrome, Frontotemporal dementia with parkinsonism linked to chromosome 17a, Gerstmann-Sträussler-Scheinker disease, Hallervorden-Spatz disease, Myotonic dystrophy, Niemann-Pick disease, type C, Non-Guamanian motor neuron disease with neurofibrillary tangles, Pick's disease, Postencephalitic parkinsonism, Prion protein cerebral amyloid angiopathy, Progressive subcortical gliosis, Progressive supranuclear palsy, Subacute sclerosing panencephalitis and Tangle only dementia.

In a further aspect the present invention provides a method for measuring or detecting tau in a sample, comprising contacting the sample with an antigen binding site of the invention as described herein and measuring or detecting binding of the antibody or antigen-binding fragment to tau. The sample may comprise one or more cells expressing tau. In some examples, the sample comprises cells such as neurons, glial cells or tissue.

In a further aspect, the present invention provides an apparatus configured to perform any one or more of the methods described herein. The apparatus may comprise any one or more of the following: an antibody delivery device configured to deliver an antibody to a subject, an acoustic energy emitter configured to emit acoustic energy for delivery to a region of the brain of the subject, a microbubble delivery device configured to deliver microbubbles to a region of the brain of the subject for disrupting the blood-brain barrier, and a controller that may control any one or more of the antibody delivery device, the acoustic energy emitter, and the microbubble delivery device. The apparatus may be used in conjunction with an imaging device, such as an MRI device, a positron emission tomography (PET) device, a computerized tomography (CT) or computerized axial tomography (CAT) device, or an ultrasound device. The apparatus may also be used in conjunction with an imaging contrast agent delivery device configured to deliver an imaging contrast agent to a region of the brain of the subject to aid in imaging of the brain by the imaging device. The imaging device and the imaging contrast agent may be controlled by the controller.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. AB1 specificity and binding is retained in all antibody formats (A) Gel electrophoresis and Coomassie staining of purified antibody formats shows that the IgG1 and IgG2a isotypes of AB1 are approximately 156 kDa in size, the Fab is approximately 52 kDa in size and the scFv is approximately 29 kDa in size. (B) The ability of AB1 in the different formats to bind to full-length human tau was assessed using ELISA and compared to a positive control antibody, tau5, and a negative control antibody, anti-Myc. AB1 in all formats was demonstrated to bind to tau. (C) Single-cycle kinetics of AB1 to full-length human tau was determined using surface plasmon resonance. The K_{D} of AB1 IgG1 and IgG2a to tau was 407 and 381 nM respectively. This was consistent with the Fab (298 nM) and scFv (460 nM). (D) The molecular weight (MW), binding affinity (K_{D}) and activity for each of the AB1 antibody formats.

FIG. 2. AB1 IgG displays increased delivery to the brain compared to the smaller antibody formats in vivo. (A) Representative images of Alexa Fluor 647-conjugated AB1 treated pR5 mice with and without SUS at 10, 35 and 60 min post-treatment using a Bruker In Vivo MS FX Pro optical imaging system. SUS was demonstrated to enhance the fluorescence intensity of all antibody formats in the brain (B) The mean fluorescence counts within the outlined region of interest shown in (A) were reported as a logarithmic scale following the subtraction of the mean of SUS-only controls. At 60 min post-treatment, the fluorescence intensity of the IgGs was significantly greater than that of the scFv and Fab in either the SUS-treated group (*P<0.0001) or the non-SUS treated group (^{#}P<0.0001). (Mean±SEM; One-way ANOVA with Tukey's multiple comparisons test.)

FIG. 3: AB1 IgG displays increased delivery to the brain compared to the smaller antibody formats post-perfusion. (A) Representative images of perfused brains from Alexa Fluor 647-conjugated AB1 treated mice 60 min post-treatment using a Bruker In Vivo MS FX Pro optical imaging system with a 630 nm excitation and a 700 nm emission filter. The fluorescence intensity of the larger antibodies was greater than that of the smaller formats with or without SUS. The fluorescence intensity of all antibody formats was enhanced when delivery was combined with SUS. The concentration of AB1 in the brain (B) and serum (C) of treated mice was estimated by comparing the fluorescence intensity with that of control brain and serum samples spiked with known concentrations of Alexa Fluor 647-conjugated AB1. (B) SUS treatment increased the mean concentration of all formats in the brain (11-fold for IgG1, 19-fold for IgG2a, 30-fold for Fab and 20-fold for scFv). Furthermore, following SUS, the concentration of the IgG2a was significantly increased compared to the scFv (**P<0.01) and Fab (^{#}P<0.05). (C) No significant difference was observed in concentration of antibody in the serum between mice treated either with or without SUS. The serum concentration of the IgGs and Fab were significantly higher than that of the scFv, either in the SUS-treated group (^{##}P<0.01) or in the non-SUS treated group (***P<0.001). One-way ANOVA with Tukey's multiple comparisons test.

FIG. 4: Wide-spread brain delivery of AB1 is detected after SUS. Fluorescence imaging of coronal sections at the dorsal hippocampus from Alexa Fluor 647-conjugated AB1 treated mouse brains labelled with the neuron-specific antibody, NeuN (green), revealed widespread brain delivery of all antibody formats (magenta) only after SUS treatment, with levels of the IgG1 and IgG2a increased compared to the Fab and scFv. Partially diffused antibody was also observed in vessels (arrow). Areas such as the thalamus, hippocampus and periventricular space consistently demonstrated an increase in antibody uptake compared to other structures. Scale bar=1 mm.

FIG. 5: The AB1 isotype is important for neuronal uptake. Confocal imaging of the somatosensory cortex of SUS-treated mice revealed that AB1 in the IgG2a, Fab or scFv format (magenta) localized within neurons labelled with NeuN (green) (arrow), whereas IgG1 (magenta) was primarily localized extracellularly and within vessels. (DAPI shown as blue). Scale bar=50 μm.

### DETAILED DESCRIPTION OF THE EMBODIMENTS

It will be understood that the invention disclosed and defined in this specification extends to all alternative combinations of two or more of the subject features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

Microtubule-associated protein tau is an attractive therapeutic target for the treatment of tauopathies such as Alzheimer's disease. Its aggregation strongly correlates with disease progression and is considered a key mediator of neuronal toxicity. Delivery of therapeutics to the brain, however, is extremely inefficient due to their limited ability to cross the blood-brain barrier (BBB). Furthermore, tau is predominantly localized intraneuronally in Alzheimer's disease, whereas in primary tauopathies, tau also aggregates in glial cells. An anti-tau therapeutic that can be internalized by neurons or glial cells may be advantageous. As identified by the inventors, one such approach that can facilitate the passing of molecules through the BBB is scanning ultrasound. Scanning ultrasound (SUS) is a non-invasive technique which transiently opens the BBB to allow peripherally delivered molecules to enter the brain. The blood-brain-barrier structure surrounds blood vessels in the brain and prevents most molecules in the blood from entering the brain and such potential adverse effects. Conversely, the blood-brain-barrier prevents the movement or clearance of molecules in the brain from entering into the peripheral circulation. In one aspect, the invention allows for a temporary increase in the permeability of the blood-brain barrier thereby allowing the natural function of the blood-brain barrier to be restored after a period of time.

The present inventors have set out to compare brain and neuronal uptake of different subtypes of tau antibody with or without a source of acoustic energy. In particular, the inventors have compared the AB1 subtype scFv to that of a larger fragment antigen binding (Fab) and full-sized murine antibodies, including the IgG1 and IgG2a isotypes, to elucidate the importance of antibody size, binding-affinity and Fc-mediated receptor binding for neuronal uptake. This has been conducted in the presence or absence of a source of acoustic energy to determine its effect on neuronal uptake.

The present inventors show for the first time that, surprisingly, despite its much larger size the IgG type is superior in regards to its delivery to the brain than an scFv. Furthermore, the inventors demonstrate that SUS is a valuable tool to increase the concentration of therapeutic antibodies between 29 and 156 kDa in the brain. These findings have therapeutic implications for the treatment of tauopathies including Alzheimer's disease as they underlie the potential for restoration or improvement of cognitive and/or memory function.

The inventors' findings are surprising because the full length subtypes of the antigen binding sites or proteins described herein demonstrate uptake by neurons, facilitated by the presence of scanning ultrasound (SUS).

An advantage of the present invention is that the antigen binding sites of the invention whether used alone, or in combination with an application of acoustic energy, are advantageous for the treatment of neurological diseases due to reduced clearance, enhanced activity and/or increased concentration in the brain.

General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 56 to 65” will be understood in an inclusive manner, i.e., the region comprises a sequence of amino acids as numbered 56, 57, 58, 59, 60, 61, 62, 63, 64 and 65 in a specified sequence.

Selected Definitions

The protein tau (Microtubule-associated protein tau, Neurofibrillary tangle protein, Paired helical filament-tau, PHF-tau) is predominantly a neuronal microtubule-associated protein and functions as a scaffolding protein and also to promote tubulin polymerization and stabilize microtubules. Several isoforms are found in the human brain, the longest isoform comprising 441 amino acids (2 amino-terminal inserts, 4 microtubule-binding domains). Tau and its properties are also described by Reynolds, C H. et al., J. Neurochem. 69 (1997) 191-198.

The term “tau” as provided herein includes any of the tau protein naturally occurring forms, homologs or variants that maintain the activity of tau (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form.

For the purposes of nomenclature only and not a limitation, an exemplary amino acid sequence of human tau is SEQ ID NO: 33 and variants thereof having at least or about 85%, 90%, 91%, 92%, 93%, 94%, 95%, 96%, 97%, 98% or 99% sequence identity. Exemplary non-human tau polypeptides include, but are not limited to, mouse, rat, pig, cow, rhesus macaque, and dog tau, and variants thereof having at least or about 85%, 90%, 91%, 92%, 93%, 94%, 95% 97%, 98% or 99% sequence identity thereto.

The term “tau” according to the invention encompasses the longest isoform of human tau, comprising 441 amino acids (SEQ ID NO: 33), fragments thereof, or any of the alternate isoforms described herein.

The term “aggregated tau” or “tau aggregation” according to the invention encompasses the aggregated form of the longest isoform of human tau, comprising 441 amino acids or any of the alternate isoforms described herein.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified,” it is meant that the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding site” is used interchangeably with “antigen binding domain” and shall be taken to mean a region of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv. Alternatively, the antigen binding domain may be in the context of an entire antibody.

For the purposes for the present disclosure, the term “antibody” includes a protein capable of specifically binding to one or a few closely related antigens (e.g., tau) by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains covalently linked and two light chains. A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH1 which is ˜330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html).

The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 are not generally involved in antigen binding.

“Framework regions” (FRs) are those variable region residues other than the CDR residues. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL.

As used herein, the term “Fv” shall be taken to mean any protein, whether comprised of multiple polypeptides or a single polypeptide, in which a VL and a VH associate and form a complex having an antigen binding domain, i.e., capable of specifically binding to an antigen. The VH and the VL which form the antigen binding domain can be in a single polypeptide chain or in different polypeptide chains. Furthermore, an Fv of the invention (as well as any protein of the invention) may have multiple antigen binding domains which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody.

A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of an antigen binding site or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “specifically binds” or “binds specifically” shall be taken to mean that an antigen binding site of the invention reacts or associates more frequently, more rapidly, with greater duration and/or with greater affinity with a particular antigen or cell expressing same than it does with alternative antigens or cells. For example, an antigen binding site binds to one tau isoform (e.g., htau) with materially greater affinity (e.g., 1.5 fold or 2 fold or 5 fold or 10 fold or 20 fold or 40 fold or 60 fold or 80 fold to 100 fold or 150 fold or 200 fold) than it does to other tau isoforms. In an example of the present invention, an antigen binding site that “specifically binds” to the tau isoform 2, tau441, (preferably human) with an affinity at least 1.5 fold or 2 fold or greater (e.g., 5 fold or 10 fold or 20 fold or 50 fold or 100 fold or 200 fold) than it does to another tau isoform, such as isoforms 1, 3, 4, 5 or 6 of human tau. In other words, an antigen binding site as described herein may bind to a tau protein with two-amino terminal inserts with an affinity at least 1.5 fold or 2 fold or greater (e.g., 5 fold or 10 fold or 20 fold or 50 fold or 100 fold or 200 fold) than it does to any other tau isoform. Generally, but not necessarily, reference to binding means specific binding, and each term shall be understood to provide explicit support for the other term.

In any aspect, an antigen binding site of the present invention does not detectably bind a tau isoform other than isoform 2. In another embodiment, the antigen binding site of the present invention does not detectably bind a tau isoform other than isoform 2N4R.

As used herein, the term “does not detectably bind” shall be understood to mean that an antigen binding site, e.g., an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using biosensor analysis (e.g. Biacore) in which the antigen binding site is immobilized and contacted with an antigen.

As used herein, the term “does not significantly bind” shall be understood to mean that the level of binding of an antigen binding site of the invention to a polypeptide is not statistically significantly higher than background, e.g., the level of binding signal detected in the absence of the antigen binding site and/or in the presence of a negative control protein (e.g., an isotype control antibody) and/or the level of binding detected in the presence of a negative control polypeptide. The level of binding is detected using biosensor analysis (e.g. Biacore) in which the antigen binding site is immobilized and contacted with an antigen.

As used herein, the term “epitope” (syn. “antigenic determinant”) shall be understood to mean a region of tau to which an antigen binding site comprising an antigen binding domain of an antibody binds. Unless otherwise defined, this term is not necessarily limited to the specific residues or structure to which the antigen binding site makes contact. For example, this term includes the region spanning amino acids contacted by the antigen binding site and 5-10 (or more) or 2-5 or 1-3 amino acids outside of this region. In some examples, the epitope comprises a series of discontinuous amino acids that are positioned close to one another when antigen binding site is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

As used herein, the term “condition” refers to a disruption of or interference with normal function, and is not to be limited to any specific condition, and will include diseases or disorders.

As used herein, the term “subject” shall be taken to mean any animal including humans, for example a mammal. Exemplary subjects include but are not limited to humans and non-human primates. For example, the subject is a human.

A skilled person will understand that executive functions include a set of cognitive processes that are necessary for the cognitive control of behaviour. Executive functions include basic cognitive processes such as attentional control, cognitive inhibition, inhibitory control, working memory, and cognitive flexibility. Higher order executive functions require the simultaneous use of multiple basic executive functions and include planning and fluid intelligence (i.e., reasoning and problem solving).

A skilled person will understand that a motor skill includes a learned ability to cause a predetermined movement outcome with maximum certainty. Motor learning is the relatively permanent change in the ability to perform a skill as a result of practice or experience. Performance is an act of executing a motor skill. The goal of motor skills is to optimize the ability to perform the skill at the rate of success, precision, and to reduce the energy consumption required for performance. Continuous practice of a specific motor skill will result in a greatly improved performance.

Antibodies

In one example, an antigen binding site or tau-binding protein as described herein according to any example is an antibody.

Methods for generating antibodies are known in the art and/or described in Harlow and Lane (editors) Antibodies: A Laboratory Manual, Cold Spring Harbor Laboratory, (1988). Generally, in such methods tau (e.g., htau) or a region thereof (e.g., an extracellular region) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (mAbs).

Monoclonal antibodies are one exemplary form of antibody contemplated by the present invention. The term “monoclonal antibody” or “mAb” refers to a homogeneous antibody population capable of binding to the same antigen(s), for example, to the same epitope within the antigen. This term is not intended to be limited with regard to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human antibodies, for example, which do not express murine antibodies, can also be used to generate an antibody of the present invention (e.g., as described in WO2002/066630).

Following immunization, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemistry and/or immunoassay (e.g. radioimmunoassay, enzyme immunoassay, cytotoxicity assay, plaque assay, dot immunoassay, and the like).

Alternatively, ABL-MYC technology (NeoClone, Madison Wis. 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

The antibody of the present invention may be a synthetic antibody. For example, the antibody is a chimeric antibody, a humanized antibody, a human antibody synhumanized antibody, primatized antibody, a de-immunized antibody or a composite antibody.

Antigen Binding Domain Containing Proteins

Single-Domain Antibodies

In some examples, a protein of the invention is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (Domantis, Inc., Waltham, Mass.; see, e.g., U.S. Pat. No. 6,248,516).

Diabodies, Triabodies, Tetrabodies

In some examples, a protein of the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding domain, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fv (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

The present invention also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present invention encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

The variable regions present in naturally occurring heavy chain antibodies are generally referred to as “V_{HH} domains” in camelid antibodies and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

Other Antibodies and Proteins Comprising Antigen Binding Domains Thereof

The present invention also contemplates other antibodies and proteins comprising antigen-binding domains thereof, such as:

(i) “key and hole” bispecific proteins as described in U.S. Pat. No. 5,731,168;

(ii) heteroconjugate proteins, e.g., as described in U.S. Pat. No. 4,676,980;

(iii) heteroconjugate proteins produced using a chemical cross-linker, e.g., as described in U.S. Pat. No. 4,676,980; and

(iv) Fab₃ (e.g., as described in EP19930302894).

Mutations to Proteins

The present invention also provides an antigen binding site or a nucleic acid encoding same having at least 80% identity to a sequence disclosed herein. In one example, an antigen binding site or nucleic acid of the invention comprises sequence at least about 85% or 90% or 95% or 97% or 98% or 99% identical to a sequence disclosed herein.

Alternatively, or additionally, the antigen binding site comprises a CDR (e.g., three CDRs) at least about 80% or 85% or 90% or 95% or 97% or 98% or 99% identical to CDR(s) of a V_{H} or V_{L} as described herein according to any example.

In another example, a nucleic acid of the invention comprises a sequence at least about 80% or 85% or 90% or 95% or 97% or 98% or 99% identical to a sequence encoding an antigen binding site having a function as described herein according to any example. The present invention also encompasses nucleic acids encoding an antigen binding site of the invention, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridizes under stringent hybridization conditions to a nucleic acid encoding an antigen binding site described herein. A “moderate stringency” is defined herein as being a hybridization and/or washing carried out in 2×SSC buffer, 0.1% (w/v) SDS at a temperature in the range 45° C. to 65° C., or equivalent conditions. A “high stringency” is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5° C. or within 10° C.) or equal to the Tm of a nucleic acid is considered to be high stringency. Medium stringency is to be considered to be within 10° C. to 20° C. or 10° C. to 15° C. of the calculated Tm of the nucleic acid.

The present invention also contemplates mutant forms of an antigen binding site of the invention comprising one or more conservative amino acid substitutions compared to a sequence set forth herein. In some examples, the antigen binding site comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., U.S. Pat. No. 4,554,101.

The present invention also contemplates non-conservative amino acid changes. For example, of particular interest are substitutions of charged amino acids with another charged amino acid and with neutral or positively charged amino acids. In some examples, the antigen binding site comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding site of the invention. In another example, the mutation(s) occur within a CDR of an antigen binding site of the invention.

Exemplary methods for producing mutant forms of an antigen binding site include:
mutagenesis of DNA (Thie et al., Methods Mol. Biol. 525: 309-322, 2009) or RNA (Kopsidas et al., Immunol. Lett. 107:163-168, 2006; Kopsidas et al. BMC Biotechnology, 7: 18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370: 389-91, 1994; and
site directed mutagenesis, e.g., as described in Dieffenbach (ed) and Dveksler (ed) (In: PCR Primer: A Laboratory Manual, Cold Spring Harbor Laboratories, N Y, 1995).

Exemplary methods for determining biological activity of the mutant antigen binding sites of the invention will be apparent to the skilled artisan and/or described herein, e.g., antigen binding. For example, methods for determining antigen binding, competitive inhibition of binding, affinity, association, dissociation and therapeutic efficacy are described herein.

Constant Regions

The present invention encompasses antigen binding sites and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG2a constant region.

In one example, the Fc region of the constant region mediates effector function and it has not been mutated to reduce effector function.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG2a constant region. The term “stabilized IgG2a constant region” will be understood to mean an IgG2a constant region that has been modified to reduce Fab arm exchange or the propensity to undergo Fab arm exchange or formation of a half-antibody or a propensity to form a half antibody. “Fab arm exchange” refers to a type of protein modification for human Ig2a, in which an IgG2a heavy chain and attached light chain (half-molecule) is swapped for a heavy-light chain pair from another IgG2a molecule. Thus, IgG2a molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG2a antibody dissociates to form two molecules each containing a single heavy chain and a single light chain.

In one example, a stabilized IgG2a constant region comprises a proline at position 241 of the hinge region according to the system of Kabat (Kabat et al., Sequences of Proteins of Immunological Interest Washington D.C. United States Department of Health and Human Services, 1987 and/or 1991). This position corresponds to position 228 of the hinge region according to the EU numbering system (Kabat et al., Sequences of Proteins of Immunological Interest Washington D.C. United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

Protein Production

In one example, an antigen binding site described herein according to any example is produced by culturing a hybridoma under conditions sufficient to produce the protein, e.g., as described herein and/or as is known in the art.

Recombinant Expression

In another example, an antigen binding site described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. Nos. 4,816,567 or 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's FI0 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

Isolation of Proteins

Methods for isolating a protein are known in the art and/or described herein.

Where an antigen binding site is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The antigen binding site prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Assaying Activity of an Antigen Binding Site

### Binding to Tau and Mutants Thereof

It will be apparent to the skilled artisan from the disclosure herein that antigen binding sites of the present invention bind to tau as described herein, or a peptide as described herein. Methods for assessing binding to a protein or peptide are known in the art, e.g., as described in Scopes (In: Protein purification: principles and practice, Third Edition, Springer Verlag, 1994). Such a method generally involves immobilizing the antigen binding site and contacting it with labelled antigen (tau). Following washing to remove non-specific bound protein, the amount of label and, as a consequence, bound antigen is detected. Of course, the antigen binding site can be labelled and the antigen immobilized. Panning-type assays can also be used. Alternatively, or additionally, surface plasmon resonance assays can be used.

Optionally, the dissociation constant (Kd), association constant (Ka) and/or affinity constant (K_{D}) of an immobilized antigen binding site for tau or an epitope thereof is determined. The “Kd” or “Ka” or “K_{D}” for an tau-binding protein is in one example measured by a radiolabelled or fluorescently-labelled tau ligand binding assay. In the case of a “Kd”, this assay equilibrates the antigen binding site with a minimal concentration of labeled tau or epitope thereof in the presence of a titration series of unlabelled tau. Following washing to remove unbound tau or epitope thereof, the amount of label is determined, which is indicative of the Kd of the protein.

According to another example the Kd, Ka or K_{D} is measured by using surface plasmon resonance assays, e.g., using BIAcore surface plasmon resonance (BIAcore, Inc., Piscataway, N.J.) with immobilized tau or a region thereof or immobilized antigen binding site.

In any aspect of present invention, the antigen binding site has a dissociation constant (K_{D}) of less than 460 nM, less that 410 nM, less than 400 nM, less than 390 nM, or less than 380 nM. Typically, the antigen binding site has any K_{D} as described herein.

Scanning Ultrasound

As a means to transiently open up the blood brain barrier, the present inventors have found that the application of acoustic energy such as scanning ultrasound (SUS) acts as an effective means for permitting or facilitating the delivery of the antigen binding sites described herein. Application or administration of acoustic energy may permit or facilitate the passage of an antigen binding site through the BBB such that the antigen binding site is then capable of binding to extracellular tau, i.e. tau present in an extracellular space. Further, application or administration of acoustic energy may permit or facilitate the passage of an antigen binding site through a cell membrane such that the antigen binding site is then capable of binding to intracellular tau, i.e. tau present within a cell, for example in the cytoplasm of a neuron or a glial cell. The intracellular tau may be associated with microtubules or it may not be associated with microtubules.

Ultrasound delivery is based on the concept of noninvasive delivery of focused ultrasound pulses that generally comprise a lipid or polymer shell, a stabilized gas core, and a diameter of less than 10 mm. In other words, the acoustic energy, such as ultrasound, can be directed by simple aiming techniques, such as physically orienting one or more transducers on a headpiece, thereby eliminating the complexities of electronic focusing and reduces the need for image guidance. This treatment also has the advantage of treating conditions where the precise site of therapy is not well defined. A highly focused approach is more likely to be unsuccessful or only partially cover the targeted region.

Acoustic energy, such as ultrasound, can be applied to the entire brain or a region of the brain. A region of the brain may be a hemisphere or forebrain. The region may be at least 25% by volume of the brain. The region of the brain may be one that is known to be associated with pathogenic protein deposition such as amyloid beta (Aβ). The particular regions of the brain to be targeted for effective treatment will differ depending on the disease. For example, for Alzheimer's disease the areas that may be targeted include the hippocampus, temporal lobe and/or basal forebrain, more specifically, the hippocampus, mamillary body and dentate gyrus, posterior cingulate gyrus, and temporal lobe. For Frontotemporal Dementia the brain region to be targeted includes the cortex. For Amyotrophic Lateral Sclerosis the region to be targeted includes the spinal cord, motor cortex, brain stem.

Identifying a region of the brain to which acoustic energy is applied may include determining a volume of the brain on the basis of symptoms displayed by the subject, typically clinically observable or biochemically detectable symptoms, or determining a volume of the brain on the basis of a known association with a neurodegenerative disease, in particular those associated with protein oligomers, aggregates or deposits, or determining a volume of the brain including a volume surrounding an site having extracellular protein in a pathogenic form, such as oligomers, an aggregate or deposit.

The focus of the acoustic energy source, typically an scanning ultrasound transducer, may be moved in a pattern with space between the subject sites of application over a region of the brain as described herein or the entire brain. The focus may be moved by a motorised positioning system. In a preferred form, the methods of the invention involve the application of focussed ultrasound to a plurality of locations in the brain. The focussed ultrasound may be applied at 2, 3, 4, 5, 6, 7, 8, 9, 10 or more locations in the brain or on each hemisphere.

It is also contemplated that any disease, condition or syndrome that is a consequence of or associated with aggregation or deposition of tau proteins in the brain, may be treated by a method of the invention. In addition, a symptom of a disease, condition or syndrome that is a consequence of or associated with aggregation or deposition of proteins in the brain, may be reduced in severity or incidence by a method of the invention.

Increasing the permeability of the blood-brain barrier can be promoted by various agents. These agents are based on the principle that biologically inert and preformed microbubbles, with either a lipid or polymer shell, a stabilized gas core, and a diameter of less than 10 um, can be systemically administered and subsequently exposed to noninvasively delivered focused ultrasound pulses.

In an embodiment of the invention, scanning ultrasound may be combined with microbubbles to disrupt the blood-brain barrier (BBB) which is achieved by mechanical interactions between the microbubbles and the blood vessel wall as pulsed focused ultrasound is applied, resulting in cycles of compression and rarefaction of the microbubbles. This leads to a transient disruption of tight junctions and the uptake of blood-borne factors by the brain. Microbubbles within the target volume become “acoustically activated” by what is known as acoustic cavitation. In this process, the microbubbles expand and contract with acoustic pressure rarefaction and compression over several cycles. This activity has been associated with a range of effects, including the displacement of the vessel wall through dilation and contraction. More specifically, the mechanical interaction between ultrasound, microbubbles, and the vasculature transiently opens tight junctions and facilitates transport across the BBB.

The microbubble agent can be any agent known in the art including lipid-type microspheres or protein-type microspheres or a combination thereof in an injectable suspension. For example, the agent can be selected from the group consisting of Octafluoropropane/Albumin (Optison), a perflutren lipid micro sphere (Definity), Galactose-Palmitic Acid microbubble suspension (Levovist) Air/Albumin (Albunex and Quantison), Air/Palm itic acid (Levovist/SHU508A), Perfluoropropane/Phospholipids (MRX115, DMP115), Dodecafluoropentane/Surfactant (Echogen/QW3600), Perfluorobutane/Albumin (Perfluorocarbon exposed sonicated dextrose albumin), Perfluorocarbon/Surfactant (QW7437), Perfluorohexane/Surfactant (Imagent/AF0150), Sulphur hexafluoride/Phospholipids (Sonovue/BR1), Perfluorobutane/Phospholipids (BR14), Air/Cyanoacrylate (Sonavist/SHU563A), and Perfluorocarbon/Surfactant (Sonazoid/NC100100).

The microbubble agent may be provided as a continuous infusion or as a single bolus dose. A continuous infusion of microbubble, preferably provided over the duration of the acoustic energy application, would be preferred. Typically, the microbubble agent is delivered intravenously through the systemic circulation. For methods of the invention that include the use of an agent such as a microbubble or other cavitation based promotion of blood-brain barrier permeability, the agent may be localized at, or near, or in a region that is targeted with the ultrasound such that the potential of unwanted damage from cavitation effects is minimised.

The applying step, for the delivery of acoustic energy, may comprise the delivery of acoustic energy from an acoustic energy source through a fluid coupler applied directly to the head of the subject. In this application, the fluid coupler may be applied to only one side or aspect of the subject's head. The head may be an unmodified head or a head with a surgically created window in the skull-the fluid coupler being in contact with the window. The acoustic energy may be generated by an unfocused acoustic energy transducer or a phased array acoustic energy transducer (i.e., focused acoustic energy). Significantly, the phased array acoustic energy transducer may be a diagnostic phased array. Diagnostic phased arrays are generally of lower power and are commonly available. The fluid coupler may comprise a contained volume of fluid (e.g., about 50 cc, about 100 cc, about 200 cc, about 400 cc, about 500 cc, about 600 cc or about 1 litre). The fluid may be, for example, water, acoustic energy gel, or a substance of comparable acoustic impedance. The fluid may be contained in a fluid cylinder with at least a flexible end portion that conforms to the subject's head. In other embodiments, the contained volume of fluid may be a flexible or elastic fluid container.

Increased permeability of the blood-brain barrier may be determined by any suitable imaging method. Preferably, the imaging method is MRI, an optical imaging method, positron emission tomography (PET), computerized tomography (CT) or computerized axial tomography (CAT) or ultrasound. If a level of acoustic energy is applied, the increased permeability of the blood-brain barrier could then be determined by any one of the methods described herein and an increased level of acoustic energy could be subsequently applied until the permeability of the bloodbrain barrier had increased to a clinically relevant level. The permeability of the BBB may also be determined by a number of known techniques including injection with Evans blue dye that binds to albumin, a protein that is normally excluded from the brain.

Any ultrasound parameters that result in clinically safe application of acoustic energy are useful in the invention. Typically, the ultrasound parameters that are preferred as those that result in an increase the permeability of the blood-brain barrier, or activate microglia phagocytosis. Various ultrasound parameters can be manipulated to influence the permeability increase in the blood-brain barrier and these include pressure amplitude, ultrasound frequency, burst length, pulse repetition frequency, focal spot size and focal depth. Several parameters are now described that are useful in a method of the invention.

Focal spot size useful in a method of the invention includes about a 1 mm to 2 cm axial width. Typically, the focal spot size has an axial width of about 1 mm to 1.5 cm, preferably 1 mm to 1 cm, even more preferably 1 mm to 0.5 cm. The length of the focal spot may be about 1 cm to as much as about 15 cm, preferably 1 cm to 10 cm, even ore preferably 1 cm to 5 cm. The focal size useful in a method of the invention is one that allows an increase in the permeability of the blood-brain barrier of the subject.

The focal depth of the ultrasound generally depends on the areas of the brain affected by the disease. Therefore, the maximum focal depth would be the measurement from the top of the brain to the base, or about 10 to about 20 cm. Focal depth could be altered by electronic focusing, preferably by using an annular array transducer. The focal depth allows application to the cortical layer which, for example, may be up to 4 cm deep.

Typically the ultrasound is applied in continuous wave, burst mode, or pulsed ultrasound. Preferably the ultrasound is applied in burst mode, or pulsed ultrasound. Pulse length parameters that are useful in a method the invention include between about 1 to about 100 milliseconds, preferably the pulse length or burst length is about 1 to about 20 milliseconds. Exemplary burst mode repetition frequencies can be between about 0.1 to 10 Hz, 10 Hz to 100 kHz, 10 Hz to 1 kHz, 10 Hz to 500 Hz or 10 Hz to 100 Hz.

The duty cycle (% time the ultrasound is applied over the time) is given by the equation duty cycle=pulse lengthxpulse repetition frequency×100. Typically, the duty cycle is from about 0.1% to about 50%, about 1% to about 20%, about 1% to about 10%, or about 1% to about 5%.

The ultrasound pressure useful in a method of the invention is the minimum required to increase the permeability of the blood-brain barrier. The human skull attenuates the pressure waves of the ultrasound which also depends on the centre frequency of the transducer, with lower centre frequencies of the ultrasound transducer causing better propagation and less attenuation. A non-limiting example of ultrasound pressure is between 0.1 MPa to 3 MPa, preferably about 0.4 or 0.5 MPa. Typically this pressure is applied to the skull, i.e. transcranially. The mechanical index characterises the relationship between peak negative pressure amplitude in situ and centre frequency with mechanical index=Pressure (MPa)/sqrt centre frequency (MHz) if this mechanical index was free from attenuation/measured from within the skull, the mechanical index would be between about 0.1 and about 2, preferably about 0.1 to 1 or 0.1 to 0.5.

A non-limiting example of a system that is able to open the blood-brain barrier is the TIPS system (Philips Research). It consists of a focused ultrasound transducer that generates a focused ultrasound beam with a centre frequency of 1-1.7 MHz focal depth of 80 mm, active outer diameter 80 mm, active inner diameter 33.5 mm which is driven by a programmable acoustic signal source within the console and attached to a precision motion assembly. An additional example of a system that is able to generate an ultrasound beam suitable for blood-brain barrier disruption is the ExAblate Neuro (Insightec) system. Suitable parameters for blood-brain barrier opening in humans such as centre frequency and microbubble dosage may be different to that in mice.

For any of the method or apparatus of the invention, the ultrasound transducer may have an output frequency of between 0.1 to 10 MHz, or 0.1 to 2 MHz. The ultrasound may be applied for a time between 10 milliseconds to 10 minutes. The ultrasound may be applied continuously or in a burst mode.

Image contrast agents, used in any methods of the invention, may be selected from the group consisting of magnetic resonance contrast agents, x-ray contrast agents (and x-ray computed tomography), optical contrast agents, positron emission tomography (PET) contrast agents, single photon emission computer tomography (SPECT) contrast agents, or molecular imaging agents. For example, the imaging contrast agent may be selected from the group consisting of gadopentetate dimeglumine, Gadodiamide, Gadoteridol, gadobenate dimeglumine, gadoversetamide, iopromide, lopam idol, Ioversol, or Iodixanol, and lobitridol.

The frequency of application of the ultrasound would generally depend on patient severity. The parameters of the ultrasound and the treatment repetition are such that there is an increase in permeability of the blood-brain barrier but preferably wherein there is no, or clinically acceptable levels of, damage to parenchymal cells such as endothelial or neuronal damage, red blood cell extravasation, haemorrhage, heating and/or brain swelling. Any method of the invention may further include performing magnetic resonance imaging on a subject comprising the steps of (a) administering a magnetic resonance contrast agent to a subject through the blood-brain barrier using any of the methods of the invention and performing magnetic resonance imaging on said subject. In this context the use of magnetic resonance imaging is to confirm the increase in permeability of the blood-brain barrier and not to locate the presence of a pathogenic protein.

Another embodiment of the invention involves providing an imaging contrast agent to the whole brain including the steps of administering an imaging contrast agent into the bloodstream of said subject; and applying ultrasound to the brain of said subject to open the bloodbrain barrier to allow the imaging contrast agent to cross the blood-brain barrier. The imaging contrast agent can be administered to the subject simultaneously or sequentially with the application of the ultrasound. In this embodiment the sequential administration of the contrast agent can be prior to or post application of the ultrasound such as SUS. In a preferred embodiment, any of the agents described herein may be administered to the bloodstream between 0 to 4 hours, between 2 to 4 hours or between 3-4 hours after ultrasound treatment using one of the methods of the invention. Preferably, the agents described herein are co-delivered.

### Conditions to be Treated

The antigen binding sites of the present invention are useful in the treatment or prevention of any condition associated, or caused by, the presence, over-expression or accumulation of tau, also referred to as tau deposits, aggregates or plaques herein.

In Alzheimer's disease other neurodegenerative diseases, the deposition of aggregates enriched in certain tau isoforms has been reported. When misfolded, this otherwise very soluble protein can form extremely insoluble aggregates that contribute to a number of tauopathies. Tau protein has a direct effect on the breakdown of a living cell caused by tangles that form and block nerve synapses. Tangles are clumps of tau protein that stick together and block essential nutrients that need to be distributed to cells in the brain, causing the cells to die.

“Tauopathies” are a class of neurodegenerative disorders resulting from the pathological function of tau, primarily the pathological aggregation of tau into filaments such as paired helical filaments (PHF) and eventually into aggregates such as neurofibrillary tangles (NFT). A “tauopathy” one of the class of neurodegenerative disorders resulting from the pathological function of tau, primarily the pathological aggregation of tau into neurofibrillary tangles (NFT). Examples of tauopathies include Alzheimer's disease, Amyotrophic lateral sclerosis/parkinsonism-dementia complex, Argyrophilic grain dementia, Corticobasal degeneration, Creutzfeldt-Jakob disease, Dementia pugilistica, Diffuse neurofibrillary tangles with calcification, Down's syndrome, Frontotemporal dementia with parkinsonism linked to chromosome 17a, Gerstmann-Sträussler-Scheinker disease, Hallervorden-Spatz disease, Myotonic dystrophy, Niemann-Pick disease, type C, Non-Guamanian motor neuron disease with neurofibrillary tangles, Pick's disease, Postencephalitic parkinsonism, Prion protein cerebral amyloid angiopathy, Progressive subcortical gliosis, Progressive supranuclear palsy, Subacute sclerosing panencephalitis and Tangle only dementia.

The human Tau (htau) protein can occur in the brain in six alternatively spliced isoforms. The longest human Tau isoform, htau40 (441 aa) (NCBI sequence reference NP_005901), comprises an amino-terminal projection domain (PD; also known as Tau projection domain or projection domain of Tau), followed by a microtubule binding domain (MTB) with four repeats and a carboxy-terminal tail. The amino-terminal projection domain of Tau protrudes from the microtubule surface when the Tau protein is bound to microtubules.

htau40 can also be referred to as 2N4R as it contains 2 amino-terminal inserts (2N) and 4 microtubule-binding repeats (4R). The two amino-terminal inserts are encoded by two alternatively spliced exons, E2 and E3, and encode 29 amino acids each. The various isoforms of the Tau protein arise from alternative splicing of exon 2, 3 and 10. The isoforms differ in either 0, 1 or 2 inserts of the 29 amino acid amino-terminal part and three or four microtubule-binding repeats. The isoforms of human Tau are summarised below:

The 0N3R isoform is 352 amino acids in length (NCBI sequence reference NP_058525.1), with the amino-terminal projection domain being 139 amino acids.

The 0N4R isoform is 383 amino acids in length (NCBI sequence reference NP_058518.1), with the amino-terminal projection domain being 139 amino acids.

The 1N3R isoform is 381 amino acids in length, with the amino-terminal projection domain being 168 amino acids.

The 1N4R isoform is 412 amino acids in length, with the amino-terminal projection domain being 168 amino acids.

The 2N3R isoform is 410 amino acids in length, with the amino-terminal projection domain being 197 amino acids.

The 2N4R isoform is 441 amino acids in length, with the amino-terminal projection domain being 197 amino acids.

The amino acid sequence of human tau isoforms can be found in publicly available databases, for example those supported by NCBI (National Center for Biotechnology Information), including GenBank®.

A subject in need of treatment may be one that exhibits impaired memory function, cognitive function or subclinical or clinical symptoms of a neurodegenerative disease. The selection of a subject for treatment may involve a screening step for identifying whether the subject is displaying impaired cognitive function, memory function or a clinical manifestation of a neurodegenerative disease. A subject in need of treatment may be one that is identified as having early, intermediate or late stage disease and in the case of Alzheimer's disease may be identified as having either diffuse Aβ oligomers or plaques.

At a clinical level, Alzheimer's disease may present a number of cognitive symptoms including mental decline, difficulty thinking and understanding, depression, hallucination, or paranoia, confusion in the evening hours, delusion, disorientation, forgetfulness, making things up, mental confusion, difficulty concentrating, inability to create new memories, inability to do simple maths, or inability to recognise common things. Behavioural symptoms may also be present and include aggression, agitation, difficulty with self-care, irritability, meaningless repetition of own words, personality changes, lack of restraint, or wandering and getting lost. Loss of loss of appetite or restlessness may also be present.

Thus, when a patient presents to a doctor with any of the above symptoms, some of the commonly used diagnostic tests include cognitive tests. Cognitive tests are used to measure and evaluate cognitive, or ‘thinking’, functions such as memory, concentration, visual-spatial awareness, problem solving, counting and language skills. Particular cognitive tests that may be used include the following.

Mini-Mental Status Examination (MMSE)

The MMSE is the most common test for the screening of dementia. It assesses skills such as reading, writing, orientation and short-term memory.

Alzheimer's Disease Assessment Scale-Cognitive (ADAS-Cog)

This 11-part test is more thorough than the MMSE and can be used for people with mild symptoms. It is considered the best brief examination for memory and language skills.

Neuropsychological Testing

A variety of tests will be used and may include tests of memory such as recall of a paragraph, tests of the ability to copy drawings or figures and tests of reasoning and comprehension.

Brain Imaging Techniques

Various brain-imaging techniques are sometimes used to show brain changes and to rule out other conditions such as tumour, infarcts (strokes—dead areas of brain tissue) and hydrocephalus (fluid on the brain); these include:

(a) Computed Tomography (CT or CAT) Scan

This technique involves taking many X-rays from different angles in a very short period of time. These images are then used to create a 3-dimensional image of the brain. CT scans are mainly used to rule out other causes of dementia such as stroke, brain tumour, multiple sclerosis or haemorrhage. They can show certain changes that are characteristic of Alzheimer's disease or other causes of dementia.

(b) Magnetic Resonance Imaging (MRI)

This technique uses powerful magnets and radiowaves to produce very clear 3-dimensional images of the brain. Currently MRI is the radiological test of choice. As well as ruling out treatable causes of dementia, MRI can reveal patterns of brain tissue loss, which can be used to discriminate between different forms of dementia such as Alzheimer's disease and frontotemporal dementia.

(c) Positron Emission Tomography (PET) and Single-Photon Emission Computerized Tomography (SPECT)

In both of these tests, a small amount of radioactive material is injected into the patient and detectors in the scanner detect emissions from the brain. PET provides visual images of activity in the brain. SPECT is used to measure blood flow to various regions of the brain.

A patient with frontotemporal dementia may show impairments in one or more of the domains of language, social cognition, perceptual-motor, executive function and complex attention without learning and memory impairment, or learning and memory impairment may be present. In Parkinson's disease motor deficits may be present with or without deficits in other domains of cognition, or deficits may be present. In Huntington's disease, motor deficits may be present without deficits in other domains of cognition, or deficits may be present. In Amyotrophic Lateral Sclerosis motor deficits may be present without deficits in other domains of cognition, or deficits may be present.

The neurodegenerative diseases to which the invention can be applied are those where pathogenic protein is extracellular and causes or contributes to the disease or a symptom thereof. The pathogenic protein may be in pathogenic form when in an altered structure such as an oligomer, an aggregate or a deposit. Alzheimer's disease, dementia with Lewy bodies, Parkinson's disease, frontotemporal lobar degeneration and British and Danish familial dementia are non-limiting examples of diseases associated with extracellular pathogenic protein. Alzheimer's disease is the most common example of these diseases in which oligomers or plaques composed of amyloid beta (Aβ) are formed in the brain. Other neurodegenerative diseases are caused by the pathological aggregation of one or more of the proteins: Amyloid beta (Aβ), amyloid fragments, amyloid precursor protein, amyloid precursor protein fragments or British peptide.

In a preferable embodiment the condition, disease or syndrome is Alzheimer's disease. In this case the subject to be treated may display impairment in the following cognitive domains including learning and memory, complex attention, executive function, perceptual motor, social cognition, and language. Alternatively, the subject may display one or more of the following symptoms: Age-associated cognitive impairment, Age-associated neuronal dysfunction not restricted to cognitive impairment, short term memory loss, inability to acquire new information, semantic memory impairments, apathy, mild cognitive impairment, language, executive or visuoconstructional problems or apraxia, long term memory impairment, irritability and aggression, and exhaustion.

Treatment as used herein refers to therapeutic treatment and also involves ameliorating a symptom associated with a disease. Therapeutic treatment can be measured by an increase or recovery in any one or more of the group consisting of cognitive function; short term memory; ability to acquire new information; semantic memory; apathy; language, executive or visuoconstructional problems or apraxia; long term memory; irritability and aggression; or exhaustion. Treatment can also be measured via reduction in the presence of pathogenic protein or a reduction in the particular forms of pathogenic protein such as protein aggregates or deposits. The presence and reduction of the pathogenic protein that can be visualised or detected by imaging techniques or biochemical techniques described herein. For example, in relation to Alzheimer's disease, treatment may relate to a reduction in a soluble or insoluble isoforms of amyloid beta (Aβ) peptide or a reduction in the number of amyloid beta (Aβ) plaques. Alternatively, the outcome of the treatment may be determined by neuropsychological or cognitive testing.

Improving memory may be determined by memory tests, typically a test administered by a clinical professional. Standardised neuropsychological tests of cognition that could be administered to test the effectiveness of the treatment include any of the following tests or one or more of its components: Neuropsychological Test Battery, Alzheimer's Disease Assessment Scale-cognitive sub scale (ADAS-cog), Mini-Mental State Examination, Severe Impairment Battery, Disability Assessment Scale for Dementia, Clinical Dementia Rating Scale Sum of Boxes, Alzheimer's Disease Cooperative Study Clinical Global Impression of Change, Wechsler Memory Scale Visual Immediate, Wechsler Memory Scale Verbal Immediate, Rey Auditory Verbal Learning Test, Wechsler Memory Digit Span, Controlled Word Association Test, Category Fluency Test, Wechsler Memory Scale Visual Delayed, Wechsler Memory Scale Verbal Delayed, Rey Auditory Verbal Learning Test, Wechsler Memory Scale, Stroop Task, Wisconsin Card Sorting Task, Trail Making Test, or any other tests of memory and executive function alone or in combination.

Various in vitro assays are also known in the art for assessing the ability of an antigen binding site to inhibit or reduce tau accumulation leading to a functional response. Assays for assessing therapeutic efficacy are described hereinabove in relation to determining neutralization by an antigen binding site, particularly in Example 1.

To determine whether an antigen binding site and/or SUS of the present invention reduces or inhibits the accumulation of tau deposits or plaques in mouse models of disease, silver staining may be used (e.g. Campbell Switzer silver staining). Further, a tau antibody may be used to quantify levels of tau following administration of antigen binding site and/or SUS. Many tau antibodies are well known in the art and may be used for the purpose of immunohistochemistry or Western blotting and include ab80579, ab75714, ab64193 (all Abcam), GTX2981 (GeneTex) and AF4394 (R&D Systems). Further, microglial phagocytosis and lysosomal uptake of tau may be determined by staining for lysosomal CD68-positive microglia and 4′,6-Diamidino-2-phenylindole (DAPI) may be used to visualize nuclei.

In an embodiment, the antigen binding site can be tested in a model of Alzheimer's disease. In this embodiment, a reduction in tau pathology such as decreases to the accumulation of tau can be assessed by measuring neurofibrillary tangle load by immunohistochemistry or any method or assay described herein.

Further, for animal models robust behavioural tests may also be conducted to determine improvements in behavioural ability in response to an antigen binding site of the present invention. For instance, the APA test may be conducted, which is a test of hippocampus dependent spatial learning in which mice learned to avoid a shock zone in a rotating arena. Additional tests include a novel object recognition (NOR) test.

Absence of brain damage may be determined by Evans Blue extravasation, absence of edemas, erythrocyte extravasation and ‘dark’ neurons as determined by Nissl staining, hematoxylin and eosin staining to determine the integrity of the cortex and the hippocampus, and absence of ischemic damage using acid fuchsin staining.

Compositions

In some examples, an antigen binding site as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing an antigen binding site into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of an antigen binding site dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding site of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, an antigen binding site of the present invention will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antigen binding site of the present invention.

WO2002/080967 describes compositions and methods for administering aerosolized compositions comprising antibodies for the treatment of, e.g., asthma, which are also suitable for administration of an antigen binding site of the present invention.

Dosages and Timing of Administration

Suitable dosages of an antigen binding site of the present invention will vary depending on the specific an antigen binding site, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding site(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding site(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding site(s), rather the present invention encompasses any amount of the antigen binding site(s) sufficient to achieve the stated result in a subject.

Kits

The present invention additionally comprises a kit comprising one or more of the following:
(i) an antigen binding site of the invention or expression construct(s) encoding same;
(ii) a source of acoustic energy, preferably scanning ultrasound (SUS);
(ii) a cell of the invention;
(iii) a complex of the invention; or
(iii) a pharmaceutical composition of the invention.

In the case of a kit for detecting tau, the kit can additionally comprise a detection means, e.g., linked to an antigen binding site of the invention.

In the case of a kit for therapeutic/prophylactic use, the kit can additionally comprise a pharmaceutically acceptable carrier.

Optionally a kit of the invention is packaged with instructions for use in a method described herein according to any example.

TABLE 1
Summary of amino acid and nucleotide sequences

| Antibody or | | SEQ ID | |
|---|---|---|---|
| protein ID | Region | NO: | Amino acid or nucleotide sequence |
| AB1 (IMGT) | LCDR1 | 1 | QSLLYSSNQKNY |
| | (protein) | | |
| | LCDR2 | 2 | WAS |
| | (protein) | | |
| | LCDR3 | 3 | QQYYGYPLT |
| | (protein) | | |
| | HCDR1 | 4 | GFSLTSYG |
| | (protein) | | |
| | HCDR2 | 5 | IWRGGS |
| | (protein) | | |
| | HCDR3 | 6 | AKNTNHRYDGYY |
| | (protein) | | |
| | VL | 7 | DIVMSQSPSSLAVSVGEKVTMSCKSSQSLLYSS |
| | (protein) | | NQKNYLAWYQQKPGQSPKLLIYWASTRESGVP |
| | | | DRFTGSGSGTDFTLTISSVKAEDLAVYYCQQYY |
| | | | GYPLTFGAGTKLELK |
| | VH | 8 | QVQLKQSGPGLVQPSQSLSITCTVSGFSLTSYG |
| | (protein) | | VHWVRQSPGKGLEWLGVIWRGGSTDYNAAFMS |
| | | | RLSITKDNSKSQVFFKMNSLQADDTAIYYCAKNT |
| | | | NHRYDGYYAMDYWGQGTSVTVS |
| | LCDR1 | 9 | CAATCCCTGCTCTACTCTTCAAATCAGAAAAAC |
| | (DNA) | | TAT |
| | LCDR2 | 10 | TGGGCAAGT |
| | (DNA) | | |
| | LCDR3 | 11 | CAGCAATATTACGGGTACCCCTTGACA |
| | (DNA) | | |
| | HCDR1 | 12 | GGGTTCAGTCTCACTTCCTATGGT |
| | (DNA) | | |
| | HCDR2 | 13 | ATATGGCGAGGGGGGTCC |
| | (DNA) | | |
| | HCDR3 | 14 | GCAAAGAATACAAACCACAGGTATGATGGATA |
| | (DNA) | | CTAC |
| | VL (DNA) | 15 | GATATAGTTATGTCTCAAAGTCCTTCAAGCCTC |
| | | | GCAGTTAGTGTTGGTGAAAAAGTAACAATGAG |
| | | | CTGCAAATCATCTCAATCCCTGCTCTACTCTTC |
| | | | AAATCAGAAAAACTATTTGGCTTGGTATCAACA |
| | | | GAAGCCCGGACAAAGTCCAAAGTTGCTCATAT |
| | | | ACTGGGCAAGTACTAGAGAGTCCGGTGTCCC |
| | | | CGATAGATTTACAGGCAGTGGCTCAGGAACCG |
| | | | ACTTCACTTTGACCATAAGTTCTGTGAAGGCA |
| | | | GAGGATTTGGCAGTTTATTATTGTCAGCAATAT |
| | | | TACGGGTACCCCTTGACATTTGGAGCCGGGAC |
| | | | TAAACTTGAGCTGAAG |
| | VH | 16 | CAAGTCCAGTTGAAGCAGAGCGGCCCCGGTC |
| | (DNA) | | TCGTCCAACCTAGCCAAAGCTTGTCCATAACTT |
| | | | GTACAGTATCAGGGTTCAGTCTCACTTCCTAT |
| | | | GGTGTGCACTGGGTCCGCCAGAGTCCTGGCA |
| | | | AGGGCCTCGAATGGCTCGGAGTAATATGGCG |
| | | | AGGGGGGTCCACTGACTATAATGCCGCTTTTA |
| | | | TGAGTAGGCTCTCTATAACTAAGGACAATTCTA |
| | | | AGAGTCAGGTCTTCTTCAAAATGAACTCCCTTC |
| | | | AGGCAGACGATACCGCTATCTATTACTGTGCA |
| | | | AAGAATACAAACCACAGGTATGATGGATACTA |
| | | | CGCTATGGATTATTGGGGTCAAGGCACCAGCG |
| | | | TCACTGTCTCC |
| | LFR1 | 17 | DIVMSQSPSSLAVSVGEKVTMSCKSS |
| | (protein) | | |
| | LFR2 | 18 | LAWYQQKPGQSPKLLIY |
| | (protein) | | |
| | LFR3 | 19 | |
| | (protein) | | TRESGVPDRFTGSGSGTDFTLTISSVKAEDLAVY |
| | | | YC |
| | LFR4 | 20 | FGAGTKLELK |
| | (protein) | | |
| | HFR1 | 21 | QVQLKQSGPGLVQPSQSLSITCTVS |
| | (protein) | | |
| | HFR2 | 22 | VHWVRQSPGKGLEWLGV |
| | (protein) | | |
| | HFR3 | 23 | TDYNAAFMSRLSITKDNSKSQVFFKMNSLQADD |
| | (protein) | | TAIYYC |
| | HFR4 | 24 | AMDYWGQGTSVTVS |
| | (protein) | | |
| | LFR1 | 25 | GATATAGTTATGTCTCAAAGTCCTTCAAGCCTC |
| | (DNA) | | GCAGTTAGTGTTGGTGAAAAAGTAACAATGAG |
| | | | CTGCAAATCATCT |
| | LFR2 | 26 | TTGGCTTGGTATCAACAGAAGCCCGGACAAAG |
| | (DNA) | | TCCAAAGTTGCTCATATAC |
| | LFR3 | 27 | ACTAGAGAGTCCGGTGTCCCCGATAGATTTAC |
| | (DNA) | | AGGCAGTGGCTCAGGAACCGACTTCACTTTGA |
| | | | CCATAAGTTCTGTGAAGGCAGAGGATTTGGCA |
| | | | GTTTATTATTGT |
| | LFR4 | 28 | TTTGGAGCCGGGACTAAACTTGAGCTGAAG |
| | (DNA) | | |
| | HFR1 | 29 | CAAGTCCAGTTGAAGCAGAGCGGCCCCGGTC |
| | (DNA) | | TCGTCCAACCTAGCCAAAGCTTGTCCATAACTT |
| | | | GTACAGTATCA |
| | HFR2 | 30 | GTGCACTGGGTCCGCCAGAGTCCTGGCAAGG |
| | (DNA) | | GCCTCGAATGGCTCGGAGTA |
| | HFR3 | 31 | ACTGACTATAATGCCGCTTTTATGAGTAGGCTC |
| | (DNA) | | TCTATAACTAAGGACAATTCTAAGAGTCAGGTC |
| | | | TTCTTCAAAATGAACTCCCTTCAGGCAGACGAT |
| | | | ACCGCTATCTATTACTGT |
| | HFR4 | 32 | GCTATGGATTATTGGGGTCAAGGCACCAGCGT |
| | (DNA) | | CACTGTCTCC |
| Human tau | Protein | 33 | MAEPRQEFEV MEDHAGTYGL GDRKDQGGYT |
| isoform 2 | | | MHQDQEGDTD AGLKESPLQT PTEDGSEEPG |
| | | | SETSDAKSTP TAEDVTAPLV DEGAPGKQAA |
| | | | AQPHTEIPEG TTAEEAGIGD TPSLEDEAAG |
| | | | HVTQARMVSK SKDGTGSDDK KAKGADGKTK |
| | | | IATPRGAAPP GQKGQANATR IPAKTPPAPK |
| | | | TPPSSGEPPK SGDRSGYSSP GSPGTPGSRS |
| | | | RTPSLPTPPT REPKKVAVVR TPPKSPSSAK |
| | | | SRLQTAPVPM PDLKNVKSKI GSTENLKHQP |
| | | | GGGKVQIINK KLDLSNVQSK CGSKDNIKHV |
| | | | PGGGSVQIVY KPVDLSKVTS KCGSLGNIHH |
| | | | KPGGGQVEVK SEKLDFKDRV QSKIGSLDNI |
| | | | THVPGGGNKK IETHKLTFRE NAKAKTDHGA |
| | | | EIVYKSPVVS GDTSPRHLSN VSSTGSIDMV |
| | | | DSPQLATLAD EVSASLAKQG |
| aa 84-97 of | Protein | 34 | TEIPEGITAEEAGI |
| the longest | | | |
| human tau | | | |
| isoform, | | | |
| tau441 | | | |
| AB1 | LCDR1 | 35 | KSSQSLLYSSNQKNYLA |
| (Chothia) | (protein) | | |
| | LCDR2 | 36 | WASTRES |
| | (protein) | | |
| | LCDR3 | 37 | QQYYGYPLT |
| | (protein) | | |
| | HCDR1 | 38 | GFSLTSY |
| | (protein) | | |
| | HCDR2 | 39 | VIWRGGS |
| | (protein) | | |
| | HCDR3 | 40 | NTNHRYDGYYAMDY |
| | (protein) | | |
| | VL | 41 | DIVMSQSPSSLAVSVGEKVTMSCKSSQSLLYSS |
| | (protein) | | NQKNYLAWYQQKPGQSPKLLIYWASTRESGVP |
| | | | DRFTGSGSGTDFTLTISSVKAEDLAVYYCQQYY |
| | | | GYPLTFGAGTKLELK |
| | VH | 42 | QVQLKQSGPGLVQPSQSLSITCTVSGFSLTSYG |
| | (protein) | | VHWVRQSPGKGLEWLGVIWRGGSTDYNAAFMS |
| | | | RLSITKDNSKSQVFFKMNSLQADDTAIYYCAKNT |
| | | | NHRYDGYYAMDYWGQGTSVTVS |
| | LCDR1 | 43 | AAATCATCTCAATCCCTGCTCTACTCTTCAAAT |
| | (DNA) | | CAGAAAAACTATTTGGCT |
| | LCDR2 | 44 | TGGGCAAGTACTAGAGAGTCC |
| | (DNA) | | |
| | LCDR3 | 45 | CAGCAATATTACGGGTACCCCTTGACA |
| | (DNA) | | |
| | HCDR1 | 46 | GGGTTCAGTCTCACTTCCTAT |
| | (DNA) | | |
| | HCDR2 | 47 | GTAATATGGCGAGGGGGGTCC |
| | (DNA) | | |
| | HCDR3 | 48 | AATACAAACCACAGGTATGATGGATACTACGC |
| | (DNA) | | TATGGATTAT |
| | VL (DNA) | 49 | GATATAGTTATGTCTCAAAGTCCTTCAAGCCTC |
| | | | GCAGTTAGTGTTGGTGAAAAAGTAACAATGAG |
| | | | CTGCAAATCATCTCAATCCCTGCTCTACTCTTC |
| | | | AAATCAGAAAAACTATTTGGCTTGGTATCAACA |
| | | | GAAGCCCGGACAAAGTCCAAAGTTGCTCATAT |
| | | | ACTGGGCAAGTACTAGAGAGTCCGGTGTCCC |
| | | | CGATAGATTTACAGGCAGTGGCTCAGGAACCG |
| | | | ACTTCACTTTGACCATAAGTTCTGTGAAGGCA |
| | | | GAGGATTTGGCAGTTTATTATTGTCAGCAATAT |
| | | | TACGGGTACCCCTTGACATTTGGAGCCGGGAC |
| | | | TAAACTTGAGCTGAAG |
| | VH | 50 | CAAGTCCAGTTGAAGCAGAGCGGCCCCGGTC |
| | (DNA) | | TCGTCCAACCTAGCCAAAGCTTGTCCATAACTT |
| | | | GTACAGTATCAGGGTTCAGTCTCACTTCCTAT |
| | | | GGTGTGCACTGGGTCCGCCAGAGTCCTGGCA |
| | | | AGGGCCTCGAATGGCTCGGAGTAATATGGCG |
| | | | AGGGGGGTCCACTGACTATAATGCCGCTTTTA |
| | | | TGAGTAGGCTCTCTATAACTAAGGACAATTCTA |
| | | | AGAGTCAGGTCTTCTTCAAAATGAACTCCCTTC |
| | | | AGGCAGACGATACCGCTATCTATTACTGTGCA |
| | | | AAGAATACAAACCACAGGTATGATGGATACTA |
| | | | CGCTATGGATTATTGGGGTCAAGGCACCAGCG |
| | | | TCACTGTCTCC |
| | LFR1 | 51 | DIVMSQSPSSLAVSVGEKVTMSC |
| | (protein) | | |
| | LFR2 | 52 | WYQQKPGQSPKLLIY |
| | (protein) | | |
| | LFR3 | 53 | GVPDRFTGSGSGTDFTLTISSVKAEDLAVYYC |
| | (protein) | | |
| | LFR4 | 54 | FGAGTKLELK |
| | (protein) | | |
| | HFR1 | 55 | QVQLKQSGPGLVQPSQSLSITCTVS |
| | (protein) | | |
| | HFR2 | 56 | GVHWVRQSPGKGLEWLG |
| | (protein) | | |
| | HFR3 | 57 | TDYNAAFMSRLSITKDNSKSQVFFKMNSLQADD |
| | (protein) | | TAIYYCAK |
| | HFR4 | 58 | WGQGTSVTVS |
| | (protein) | | |
| | LFR1 | 59 | GATATAGTTATGTCTCAAAGTCCTTCAAGCCTC |
| | (DNA) | | GCAGTTAGTGTTGGTGAAAAAGTAACAATGAG |
| | | | CTGC |
| | LFR2 | 60 | TGGTATCAACAGAAGCCCGGACAAAGTCCAAA |
| | (DNA) | | GTTGCTCATATAC |
| | LFR3 | 61 | GGTGTCCCCGATAGATTTACAGGCAGTGGCTC |
| | (DNA) | | AGGAACCGACTTCACTTTGACCATAAGTTCTGT |
| | | | GAAGGCAGAGGATTTGGCAGTTTATTATTGT |
| | LFR4 | 62 | TTTGGAGCCGGGACTAAACTTGAGCTGAAG |
| | (DNA) | | |
| | HFR1 | 63 | CAAGTCCAGTTGAAGCAGAGCGGCCCCGGTC |
| | (DNA) | | TCGTCCAACCTAGCCAAAGCTTGTCCATAACTT |
| | | | GTACAGTATCA |
| | HFR2 | 64 | GGTGTGCACTGGGTCCGCCAGAGTCCTGGCA |
| | (DNA) | | AGGGCCTCGAATGGCTCGGA |
| | HFR3 | 65 | ACTGACTATAATGCCGCTTTTATGAGTAGGCTC |
| | (DNA) | | TCTATAACTAAGGACAATTCTAAGAGTCAGGTC |
| | | | TTCTTCAAAATGAACTCCCTTCAGGCAGACGAT |
| | | | ACCGCTATCTATTACTGTGCAAAG |
| | HFR4 | 66 | TGGGGTCAAGGCACCAGCGTCACTGTCTCC |
| | (DNA) | | |

### EXAMPLES

This study aimed to compare brain and neuronal uptake of the AB1 scFv, delivered with or without a source of acoustic energy (SUS), to that of a larger fragment antigen binding (Fab) and full-sized murine antibodies, including the IgG1 and IgG2a isotypes, to elucidate the importance of antibody size, binding-affinity and Fc-mediated receptor binding for neuronal uptake.

### Materials and Methods

### Reagents

Primary antibodies used in this study were as follows: NeuN (1:1,500; Millipore), tau5 (1:1,000; Millipore) and anti-Myc (1:1000; Cell Signalling Technologies). The secondary antibodies used in this study were as follows: Polyclonal rabbit anti-mouse IgG biotinylated and polyclonal goat anti-rabbit IgG biotinylated (1:500; Dako) and Alexa Fluor 488 donkey anti-rabbit (1:500; Life Technologies).

### Antibody Generation

AB1 is a mouse monoclonal antibody raised against the tau peptide TEIPEGITAEEAGI (aa 84-97 of the longest human tau isoform, tau441) and specific for the 2N isoforms of tau. An scFv was generated and is approximately 29 kDa in size. To generate both IgG1 and IgG2a isotypes, the variable heavy (VH) chain of AB1 was cloned into mAbXpress mouse IgG1 and IgG2a plasmids, and the variable light chain was cloned into a mAbXpress IgG LC kappa plasmid. IgG expression in Expi-CHO cells (ThermoFisher) and Protein A purification were conducted at the University of Queensland Protein Expression Facility. The calculated molecular weights of the IgG1 and IgG2a are 155.9 and 156.3 kDa, respectively.

To produce the Fab, purified IgG2a was digested with papain using the Pierce™ Fab Preparation Kit (Thermo Fisher Scientific). The calculated molecular weight of the Fab is 52 kDa. The scFv with a C-terminal His6 and myc tag was expressed in BL21 cells and purified. All antibody formats were stored in 1×PBS (137 mM NaCl, 2.7 mM KCl, 10 mM Na2PO4, 1.8 mM KH2PO4) at −80° C. To assess AB1 scFv and Fab generation, proteins were electrophoresed on a 10% tris-glycine SDS-PAGE gel and then stained with Coomassie blue R250 (Biorad).

### Expression and Purification of Recombinant Tau

Human and mouse tau expression and purification were performed as previously described (Liu C, et al. Journal of Biological Chemistry. 2016; 291: 8173-88).

### ELISA

The binding specificity of AB1 antibody formats was analyzed using an enzyme-linked immunosorbent assay (ELISA) as previously described (Liu C, et al. Journal of Biological Chemistry. 2016; 291: 8173-88).

### Surface Plasmon Resonance

Surface plasmon resonance measurements were conducted at the Monash Fragment Platform, Monash University, using the Biacore S200 biosensor (GE Healthcare). Biotinylated AB1 was captured on a streptavidin-coated CM5 chip. For biotinylation, AB1 IgG1 (29.5 μM), AB1 IgG2a (32.0 μM), AB1 Fab (22.9 μM) and AB1 scFv (60.4 μM) (in 137 mM NaCl, 2.7 mM KCl, 10 mM Na2HPO4, 1.8 mM KH2PO4) was added in a 1:1 ratio with EZ-link NHS-LC-LC-biotin (Thermo Scientific) and incubated at 25° C. for 1 hr. Antibodies were separated from free-unconjugated biotin using size-exclusion chromatography on a Superdex 200 10/300 GL (GE Healthcare) or Superdex 75 10/300 Increase (GE Healthcare) equilibrated in 137 mM NaCl, 2.7 mM KCl, 12 mM Na2HPO4, 1.8 mM KH2PO4, pH 7.4. Streptavidin was immobilized on the CM5 chip using amine coupling at 37° C. Antibodies were captured at 25° C., using a flowrate of 10 μL/min, in immobilization buffer 1 (137 mM NaCl, 2.7 mM KCl, 12 mM Na2HPO4, 1.8 mM KH2PO4, pH 7.4) for IgG1, IgG2a and Fab or immobilization buffer 2 (12 mM Na2HPO4, 287 mM NaCl, 2.7 mM KCl, 1.8 mM KH2PO4, 0.05% Tween-20 pH 7.4) for the scFv. tau binding experiments were run using single-cycle kinetics at 25° C. with the running buffer 12 mM Na2HPO4, 287 mM NaCl, 2.7 mM KCl, 1.8 mM KH2PO4, 0.05% Tween-20 pH 7.4. tau was injected for 120 sec at a flow rate of 40 μL/min with a dissociation time of 600s, using 8 concentrations of tau (⅓ serial dilutions from 0.0128-1,000 nM). The data were processed using Biacore S200 Evaluation Software Version 1.0, double referenced against blank injections of buffer and fit to a Steady State Affinity model using report points 4 sec before the injection end, with a 5 sec window.

### Antibody Labelling

AB1 was covalently conjugated with Alexa Fluor 647 dye (Thermo Fisher) in PBS with 0.1M sodium bicarbonate, as described previously [12]. The labelled antibodies were then separated from free dye using a Superdex 200 10/300 column (GE Healthcare) equilibrated in 1×PBS, pH 7.4, at 0.5 mL/min, and concentrated to the desired injection volumes using Amicon Ultracel 15k concentration filters (Millipore).

### Production of Microbubbles

In-house prepared microbubbles comprising a phospholipid shell and octafluoropropane gas core were used. 1,2-distearoyl-sn-glycero-3-phosphocholine (DSPC) and 1,2-distearoyl-snglycero-3-phosphoethanolamine-N-[amino(polyethylene glycol)-2000] (DSPE-PEG2000) (Avanti Polar Lipids) were mixed at a 9:1 molar ratio dissolved in chloroform (Sigma) and the chloroform solvent was evaporated under vacuum. The dried phospholipid cake was then dissolved in PBS with 10% glycerol to a concentration of 1 mg lipid/ml and heated to 55° C. in a sonicating water bath. The solution was placed in 1.5 mL glass HPLC vials and the air in the vial was replaced with octafluoropropane (Arcadophta). Microbubbles were generated on the day of the experiment by agitation in a ViaImix (Lantheus) at 4000 rpm for 40 sec.

Microbubbles were observed under a microscope to be polydispersed and under 10 μm in size at a concentration of 1-5×10⁸ microbubbles/mL.

### Mice

All animal experiments were conducted under the guidelines of the Australian Code of Practice for the Care and Use of Animals for Scientific Purposes and were approved by the University of Queensland Animal Ethics Committee (QBI/412/14/NHMRC; QBI/027/12/NHMRC). pR5 mice express 2N4R tau with the P301L mutation under the control of the mThy.1.2 promoter.

### In Vivo Antibody Delivery

For each antibody format, 6 month-old pR5 mice were randomly assigned to one of the following groups: SUS only, antibody only, or SUS and antibody combined. Five mice were used per experimental group except in the IgG2a (no SUS) group where only four mice were used. 24 hours prior to treatment, animals were anesthetized with ketamine (100 mg/kg) and xylazine (10 mg/kg), their whole body shaved and residual hair removed using hair removal cream. To avoid hindrance of infra-red detection, mice which had black or darker skin on the scalp were excluded from the study due to hindrance of infra-red detection. Immediately prior to treatment, all animals were anesthetized again and prepared as previously described (Leinenga G, Götz J. Science translational medicine. 2015; 7: 278ra33).

For the SUS-only group, mice were injected retro-orbitally with 40 μL of microbubble solution prepared as previously described (Leinenga G, Götz J. Science translational medicine. 2015; 7: 278ra33). For the antibody only and antibody plus SUS groups, 3 nanomole of each antibody and microbubble solution (40 μl) were mixed in a 29 G insulin syringe (Trumo), incubated for 2 min and then injected retro-orbitally. The maximal combined injection volume was 120 μL.

### Scanning Ultrasound

Animals which received acoustic energy in the form of SUS were placed in a head frame (Narishige) and SUS was applied to the entire brain as previously described (Leinenga G, Götz J. Science translational medicine. 2015; 7: 278ra33). Briefly, SUS was conducted using the Therapy Imaging Probe System (TIPS, Philips Research) with the following settings: 1 MHz center frequency, 0.7 MPa peak rarefactional pressure applied outside the skull, 10 Hz pulse repetition frequency, 10 ms pulse length and a 10% duty cycle. The focus of the transducer had dimensions of 1.5 mm×12 mm in the transverse and axial planes, respectively. The motorized positioning system moved the focus of the transducer array in a grid with 1.5 mm spacing between subject sites of sonication so that ultrasound was delivered sequentially to the entire brain with a 6 sec duration sonication per spot.

### In Vivo Imaging

Mice were kept under 1-2% isoflurane and were scanned every 10 min up until 1 hr posttreatment using a Bruker In Vivo MS FX Pro optical imaging system with x-ray and a 630 nm excitation and a 700 nm emission filter. Upon completion of the scans, blood was collected from the anesthetized mice using an EDTA-coated 27G syringe, after which they were transcardially perfused with PBS and their brains harvested. The dissected brains were then rescanned as above, after which the hemispheres were separated and one hemisphere snapfrozen, and the other immersion-fixed in 4% paraformaldehyde (Sigma).

### Image Analysis

Whole animal scans were analyzed using Bruker Molecular Imaging software. An ellipsoid region of interest (ROI) was drawn in the brain of each mouse at every time point posttreatment, with the calibrated unit of radiant efficiency (P/s/mm2) being reported for each ROI. Raw signal was log-transformed to improve Q-Q plot normality.

### Determination of Brain and Blood Antibody Concentrations

The snap-frozen brain hemispheres were diluted 3× in RIPA buffer, homogenized and lyzed. Alexa Fluor 647 fluorescence intensity in the soluble RIPA fraction of brain homogenate was measured using a ClarioStar Fluorescent plate reader (BMG Labtech) and compared to standard curves of Alexa Fluor 647-conjugated antibody-spiked brain homogenate. The serum concentration was calculated in the same way, except that control mouse serum was used for the standard curve.

### Histology

Fixed brain hemispheres were cryo-protected by immersion in 30% sucrose and then sectioned at 40 μm thickness on a freezing sliding microtome. For immunofluorescence sections at the dorsal hippocampus were incubated with NeuN overnight, followed by 2 hr incubation in Alexa Fluor 488-conjugated secondary antibodies. Sections were mounted on slides and stained with 1 pg/mL DAPI in PBS for 10 min, then cover-slipped with fluorescence mounting medium (Dako). Images were taken with a Diskcovery Spinning Disk confocal microscope with Nikon software.

### Statistical Analysis

Statistical analyses were performed with GraphPad Prism 7.0 software using one-way ANOVAs with Tukey's multiple comparison test. All values are given as the mean±standard error of the mean (SEM).

### Results

AB1 Antibody Size does not Impact Binding Specificity and Affinity

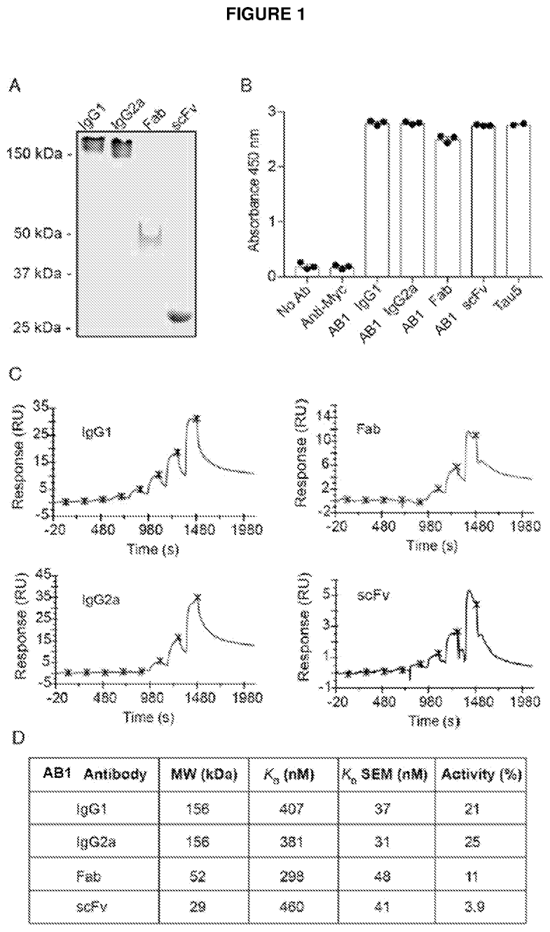

The goal of this study was to determine whether antibody properties, including size, affinity and Fc receptor binding, are important for effective brain and neuronal uptake. To achieve this, full-sized murine IgG1 and IgG2a (156 kDa), Fab (52 kDa) and scFv (29 kDa) were generated (FIG. 1A). Mouse IgG can be divided into four subclasses: IgG1, IgG2a, IgG2b and IgG3. These subclasses mediate effector functions differently due to variable specificity and affinity for Fc receptors, including the intracellular Fc receptor, TRIM21, the neonatal Fc receptor (FcRn) and the family of Fcγ receptors (FcγRIa, FcγRIII, FcγRIV and FcγRIIb). The murine IgG1 and IgG2a isotypes, equivalent to the human IgG2 and IgG1, respectively, were selected for this study as IgG1 only binds FcγRII and FcγRIII with low affinity, whereas mouse IgG2a binds to all receptors with FcγRI>FcγRIV>FcγRIII>FcγRIIb, thereby allowing us to determine if Fc receptor binding is an important property for IgG delivery across the BBB and into neurons.

To ensure that binding to tau was retained in all formats, an ELISA was performed against human tau, which revealed that antibody engineering did not negatively impact antigen binding (FIG. 1B). Furthermore, the binding affinity to tau, determined by single-cycle surface plasmon resonance, differed only slightly between the antibody formats, being within the range of 300-460 nM (FIG. 1C). Although, the percentage of each antibody that actively bound to tau differed markedly, as the IgG1 and IgG2a were 23-25% active, whereas the Fab and scFv were 11% and 4% active, respectively (FIG. 1C). This, however, is most likely caused by the biotinylation and immobilization process, which blocks the antigen binding sites of the smaller formats more readily, thereby decreasing their binding activity.

### AB1 Size Affects Delivery to the Brain

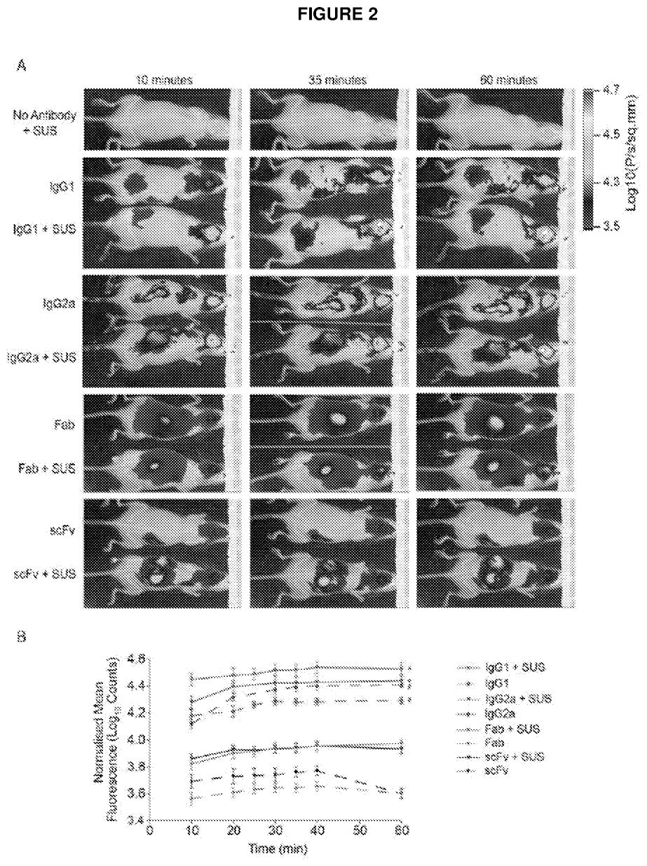

We next sought to compare the delivery of the different antibody formats to the brain with and without SUS in P301L tau transgenic pR5 mice, a strain characterized by progressive tau pathology in the hippocampus, cortex and amygdala [16]. In vivo whole-body imaging revealed that all antibody formats were detected in the brains of mice in the antibody-only treated groups (FIG. 2A). However, following SUS, brain uptake was increased for all antibody formats (FIG. 2A, B). Full-sized antibodies, however, were delivered to the brain more effectively, irrespective of whether SUS was used, compared to the scFv and Fab formats (FIG. 2B). This could be attributed to the smaller antibody fragments showing high levels in the kidneys (FIG. 2A) and bladder (data not shown), suggesting that they are being rapidly cleared from the blood through the renal system, thereby reducing the amount available for delivery to the brain. In the case of the IgGs, in both the presence and absence of SUS, levels appeared to reach a maximum at approximately 40 min post-treatment and remained constant for an additional 20 minutes (FIG. 2B). This suggests that once the IgG1 or IgG2a is delivered to the brain, these formats are not rapidly cleared. The brain levels of scFv and Fab also reached a maximum approximately 40 min post-delivery; however, they then decreased in the non-SUS group but remained constant in the SUS-treated group, suggesting that SUS plays a role in the continued delivery and/or retention of smaller antibody fragments in the brain (FIG. 2B).

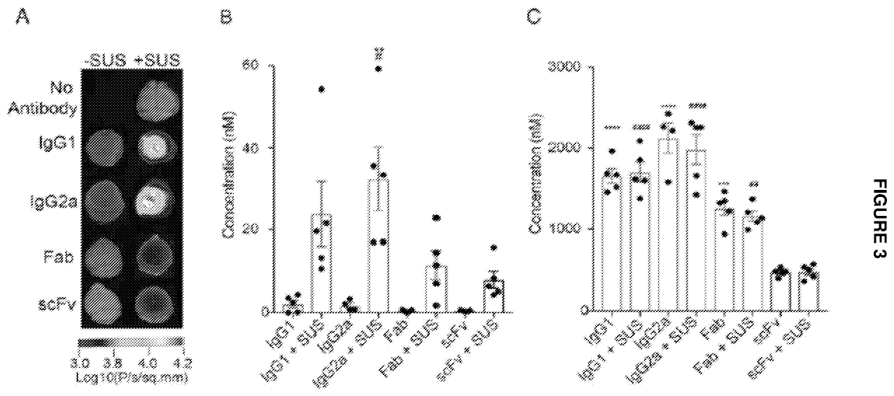

To confirm that the antibody signals detected in vivo were localized in the brain parenchyma, the treated mouse brains were perfused, thereby flushing antibody from the cerebral vasculature. Ex-vivo imaging of these brains generated data that were consistent with those obtained from the whole-body scans, with SUS-treated mice displaying increased delivery of AB1 into the brain compared to non-SUS treated animals (FIG. 3A). Furthermore, the levels of the full-sized antibodies in the brain were greater than those of the smaller fragments (FIG. 3A). Calculation of the brain antibody levels, either with or without SUS, revealed that antibodies with highest concentrations in the brain were the IgG1 (2.1 nM without SUS and 24 nM with SUS; 11-fold difference) and IgG2a (1.7 nM without SUS and 32 nM with SUS; 19-fold difference) compared to the Fab (0.39 nM without SUS and 12 nM with SUS; 30-fold difference) and scFv (0.42 nM without SUS and 8.1 nM with SUS; 20-fold difference) (FIG. 3B). In contrast, the serum levels did not differ between SUS versus no-SUS treatment (FIG. 3C). The concentration of the scFv in the serum, either with or without SUS, was significantly less than the IgGs and Fab, however, which may be attributed to the increased clearance of the scFv through the renal system.

Antibody Formats are Detected Throughout the Parenchyma after SUS

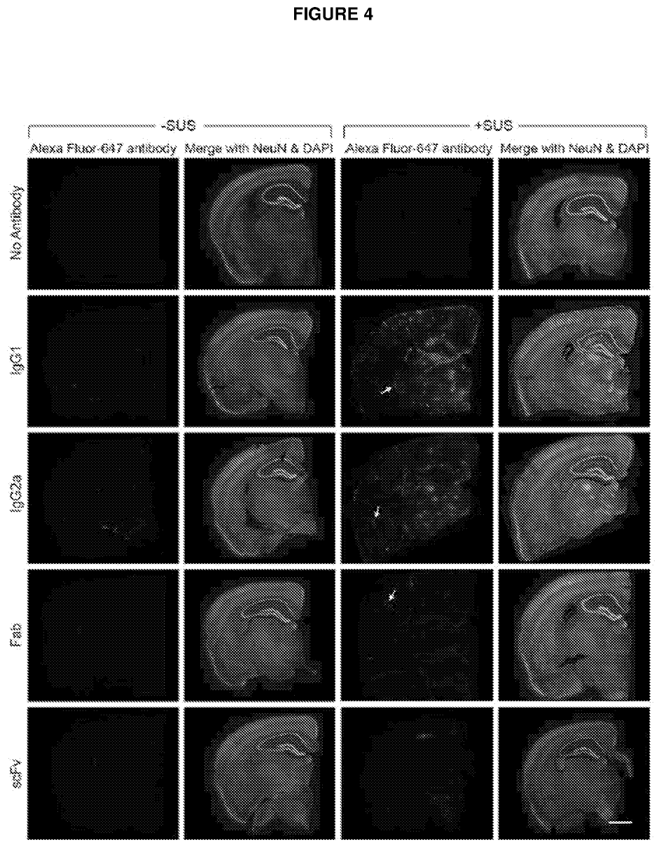

We next investigated the distribution of the delivered AB1 formats in coronal brain sections at the dorsal hippocampus. Without SUS, antibody levels were mostly undetectable, except in rare instances where IgG1 and IgG2a appeared to be visible in periventricular areas (FIG. 4). With SUS, however, all antibody formats were observed throughout the brain, with IgG1 and IgG2a levels markedly greater than those of the smaller antibody fragments (FIG. 4). In addition, partially diffused antibodies were also observed surrounding blood vessels. Interestingly, although antibody fluorescence was observed in most areas of the brain, structures such as the hippocampus, thalamus and periventricular spaces consistently showed greater antibody fluorescence (FIG. 4).

### Neuronal Uptake is Dependent on Antibody Isotype but not Size

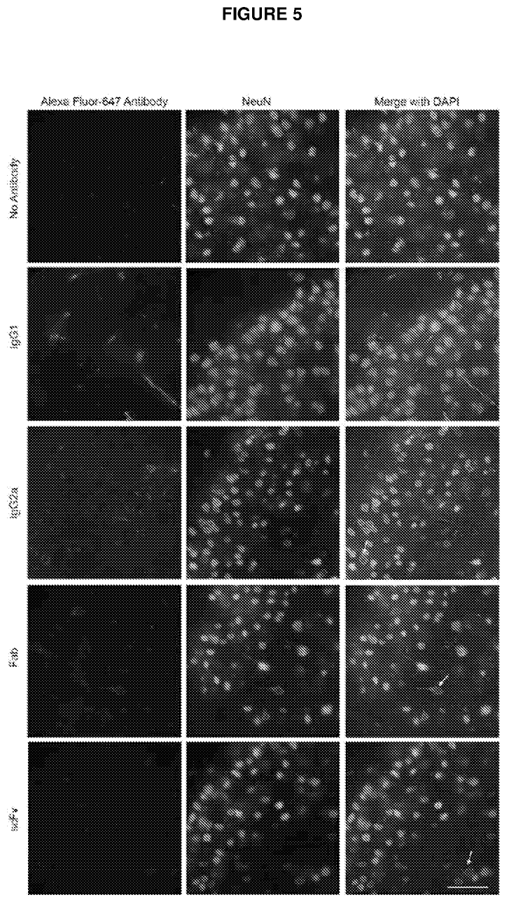

To determine if antibody size and isotype affected neuronal uptake, we investigated colocalization of AB1 with the neuron-specific antibody, NeuN, in the somatosensory cortex. Without SUS, intraneuronal AB1 was almost completely absent, with the exception of IgG2a, which showed minor uptake into several neurons in the thalamus (data not shown). With SUS, however, neuronal uptake was observed for the IgG2a, Fab and scFv formats (FIG. 5).

### Discussion

Not only is the BBB a major hurdle for the treatment of neurological disorders, but for the treatment of disorders characterized by intracellular pathogenic proteins, the cell membrane also presents as additional challenge. It is therefore imperative to establish techniques to enhance the delivery of therapeutics across the BBB and into neurons and determine the biophysical parameters which make therapeutics amenable to this transport. Using AB1 as a model antibody, we sought to investigate the impact antibody size and isotype has on the ability of AB1 to be delivered across the BBB and into neurons in the presence and absence of SUS.

Here we demonstrate that SUS not only increases the delivery of AB1 in the scFv format to the brain, but that it also increases delivery of the larger Fab and IgG formats, demonstrating SUS is an effective tool to deliver antibodies between 29 and 156 kDa. Despite the whole brain undergoing SUS, however, antibody delivery was not homogenous, with the greatest fluorescence detected in structures such as the hippocampus and thalamus, suggesting that some regions in the brain are more permeable to SUS delivery. Furthermore, all antibody formats demonstrated increased fluorescence intensity surrounding vessels suggesting the antibodies were either within or attached to the membrane of endothelial cells which line the vessel wall. These observations are consistent with the delivery of 70 kDa dextrans, approximately the average size of the antibody formats we analyzed. When delivery and permeability of these dextrans were compared to smaller dextrans of 3 kDa, a more heterogenous distribution of the larger dextrans was revealed.

Despite their large size, we observed from in vivo imaging that the AB1 IgGs had the greatest delivery to the brain either with or without SUS. This is most likely due to the increased circulating serum levels of the IgGs compared to the smaller Fab and scFv formats, which are rapidly cleared through the kidneys due to their low molecular weights. In addition, we demonstrated that the concentration of the IgG formats in the brain post-perfusion were also greater than the smaller antibody formats, confirming that the IgGs display an enhanced delivery to the brain.

In conclusion, we have demonstrated that SUS is a valuable tool for enhancing the delivery of antibodies between 29 and 156 kDa in size across the BBB and into neurons. Furthermore, although scFvs are advantageous for diagnostic imaging, an IgG would be therapeutically advantageous for the treatment of neurological diseases due to its reduced clearance, enhanced activity and increased concentration in the brain.

1. A method of delivering an antigen binding site that binds to or specifically binds to tau in a subject comprising:
administering to the subject an antigen binding site that binds to or specifically binds to tau, and
administering acoustic energy to the brain of the subject;
wherein the application of acoustic energy acts as a means to permit or facilitate the antigen binding site to pass through the blood-brain barrier (BBB) of the subject,
wherein the antigen binding site has a molecular weight greater than about 29 kDa,
thereby delivering the antigen binding site that binds to or specifically binds to tau.

2. A method of improving memory, motor skills, executive functions and/or cognitive function in a subject, the method comprising, consisting essentially of or consisting of the steps of:
administering an antigen binding site that binds to or specifically binds to tau to the subject;
identifying a region of the brain of the subject to which acoustic energy is to be applied; and
applying a clinically safe level of acoustic energy to the region, thereby saturating or substantially saturating the region with acoustic energy;
wherein the antigen binding site has a molecular weight greater than about 29 kDa,
thereby improving the memory, motor skills, executive functions and/or cognitive function in the subject.

3. A method for treating, delaying, reducing, inhibiting or preventing the accumulation or deposition of pathological tau aggregates in the central nervous system in a subject, comprising
administering an antigen binding site that binds to or specifically binds to tau to the subject;
and
administering acoustic energy to the brain of the subject,
wherein the application of acoustic energy acts as a means to permit or facilitate the antigen binding site to pass through the blood-brain barrier (BBB),
wherein the antigen binding site has a molecular weight greater than about 29 kDa,
thereby treating, delaying, reducing, inhibiting or preventing the accumulation or deposition of pathological tau aggregates in the central nervous system in a subject.

4. A method of treating a neurodegenerative condition associated with the presence, over-expression or accumulation of tau, the method comprising
administering an antigen binding site that binds to or specifically binds to tau to the subject; and
administering acoustic energy to the brain of the subject,
wherein the application of acoustic energy acts as a means to permit or facilitate the antigen binding site to pass through the blood-brain barrier (BBB),
wherein the antigen binding site has a molecular weight greater than about 29 kDa,
thereby treating a neurodegenerative condition associated with the presence, over-expression or accumulation of tau.

5. A method according to any one of claims 1 to 4, wherein the antigen binding site has a molecular weight greater than an scFv.

6. A method according to any one of claims 1 to 4, wherein the antigen binding site has a molecular weight of between about 29 and 156 kDa.

7. A method according to any one of claims 1 to 6, wherein the antigen binding site comprises a fragment crystallizable region (Fc region).

8. A method according to any one of claims 1 to 7, wherein the antigen binding site is an IgG type.

9. A method according to claim 8, wherein antigen binding site is an IgG2 isotype.

10. A method according to claim 9, wherein the antigen binding site is an IgG2a isotype.

11. A method according to any one of claims 1 to 10, wherein the antigen binding site of the invention binds to or specifically binds to human tau.

12. A method according to claim 11, wherein the human tau comprises, consists essentially of or consists of the amino acid sequence shown in SEQ ID NO: 33.

13. A method according to any one of claims 1 to 12, wherein the antigen binding site binds with higher affinity to a 2N isoform of the tau protein than any other tau isoform.

14. A method according to claim 13, wherein the antigen binding site binds with a higher affinity to the 2N isoform than the 1N or 0N isoform.

15. A method according to any one of claims 1 to 14, wherein the antigen binding site binds to or specifically binds to a human tau molecule comprising, consisting essentially of or consisting of an amino acid sequence of residues, or residues equivalent to, 84 to 97 of the human tau isoform, tau441.

16. A method according to claim 15, wherein the amino acid sequence of residues 84 to 97 of tau441 is shown in SEQ ID NO: 34.

17. A method according to any one of claims 1 to 16, wherein the antigen binding site binds to a peptide comprising, consisting essentially of or consists of the sequence: TEIPEGITAEEAGI (SEQ ID NO:34).

18. A method according to any one of claims 1 to 17, wherein the antigen binding site is not an scFv.

19. A method according to any one of claims 1 to 18, wherein tau is intracellular.

20. A method according to claim 19, wherein the intracellular tau is in a neuron or a glial cell.

21. A method according to claim 20, wherein the neuron is in the brain.

22. A method according to any one of claims 1 to 21, wherein the antigen binding site has a KD for tau less than about 460 nM, less than 450 nM, less than 410 nM, less than 400 nM or less than about 390 nM.

23. A method according to any one of claims 1 to 22, wherein the acoustic energy is ultrasound.

24. A method according to any one of claims 1 to 23, wherein the method further comprises administering microbubbles to disrupt the blood-brain barrier.

25. A method according to any one of claims 1 to 24, wherein the subject is diagnosed as having a condition or disease associated with, or caused by, a pathological form of tau.

26. A method according to claim 25, wherein the pathological form of tau is an oligomer, aggregate or deposit.

27. A method according to claim 25 or 26, wherein the subject is diagnosed as having a tauopathy.

28. A method according to claim 27, wherein the tauopathy is selected from the group consisting of Alzheimer's disease, Amyotrophic lateral sclerosis/parkinsonism-dementia complex, Argyrophilic grain dementia, Corticobasal degeneration, Creutzfeldt-Jakob disease, Dementia pugilistica, Diffuse neurofibrillary tangles with calcification, Down's syndrome, Frontotemporal dementia with parkinsonism linked to chromosome 17a, Gerstmann-Sträussler-Scheinker disease, Hallervorden-Spatz disease, Myotonic dystrophy, Niemann-Pick disease, type C, Non-Guamanian motor neuron disease with neurofibrillary tangles, Pick's disease, Postencephalitic parkinsonism, Prion protein cerebral amyloid angiopathy, Progressive subcortical gliosis, Progressive supranuclear palsy, Subacute sclerosing panencephalitis and Tangle only dementia.

29. A method according to claim 23, wherein the ultrasound is focussed or unfocussed.

30. An antigen binding site comprising an antigen binding domain of an antibody, wherein the antigen binding domain binds to or specifically binds to tau, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO:4 or 38, a CDR2 comprising a sequence at least about 80%, at least 85%, at least 90%, at least 92%, at least 95%, at least 97%, at least 99% identical to a sequence set in SEQ ID NO:5 or 39 and a CDR3 comprising a sequence at least about 80%, at least 85%, at least 90%, at least 92%, at least 95%, at least 97%, at least 99% identical to a sequence set forth in SEQ ID NO: 6 or 40;
(ii) a VH comprising a sequence at least about 95% or 96% or 97% or 98% or 99% identical to a sequence set forth in SEQ ID NO: 8 or 42;
(iii) a VL comprising a CDR1 comprising a sequence at least about 80%, at least 85%, at least 90%, at least 92%, at least 95%, at least 97%, at least 99% identical to a sequence set forth in SEQ ID NO: 1 or 35, a CDR2 comprising a sequence at least about 80%, at least 85%, at least 90%, at least 92%, at least 95%, at least 97%, at least 99% identical to a sequence set forth in SEQ ID NO: 2 or 36 and a CDR3 comprising a sequence at least about 80%, at least 85%, at least 90%, at least 92%, at least 95%, at least 97%, at least 99% identical to a sequence set forth in SEQ ID NO: 3 or 37;
(iv) a VL comprising a sequence at least about 95% identical to a sequence set forth in SEQ ID NO: 7 or 41;
(v) a VH comprising a CDR1 comprising a sequence set forth in SEQ ID NO: 4 or 38, a CDR2 comprising a sequence set forth between in SEQ ID NO: 5 or 39 and a CDR3 comprising a sequence set forth in SEQ ID NO: 6 or 40;
(vi) a VH comprising a sequence set forth in SEQ ID NO: 8 or 42;
(vii) a VL comprising a CDR1 comprising a sequence set SEQ ID NO: 1 or 35, a CDR2 comprising a sequence set forth in SEQ ID NO: 2 or 36 and a CDR3 comprising a sequence set forth in SEQ ID NO: 3 or 37;
(viii) a VL comprising a sequence set forth in SEQ ID NO: 7 or 41;
(ix) a VH comprising a CDR1 comprising a sequence set forth in SEQ ID NO: 4 or 38, a CDR2 comprising a sequence set forth between in SEQ ID NO: 5 or 39 and a CDR3 comprising a sequence set forth in SEQ ID NO: 6 or 40; and a VL comprising a CDR1 comprising a sequence set SEQ ID NO: 1 or 35, a CDR2 comprising a sequence set forth in SEQ ID NO: 2 or 36 and a CDR3 comprising a sequence set forth in SEQ ID NO: 3 or 37; or
(x) a VH comprising a sequence set forth in SEQ ID NO: 8 or 42 and a VL comprising a sequence set forth in SEQ ID NO: 7 or 41.

31. An antigen binding site according to claim 30, wherein the antigen binding domain comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:21 or 55, a FR2 comprising a sequence at least about 80%, at least 85%, at least 90%, at least 92%, at least 95%, at least 97%, at least 99% identical to a sequence set in SEQ ID NO:22 or 56, a FR3 comprising a sequence at least about 80%, at least 85%, at least 90%, at least 92%, at least 95%, at least 97%, at least 99% identical to a sequence set forth in SEQ ID NO: 23 or 57, and a FR4 comprising a sequence at least about 80%, at least 85%, at least 90%, at least 92%, at least 95%, at least 97%, at least 99% identical to a sequence set forth in SEQ ID NO: 24 or 58;
(ii) a VL comprising a FR1 comprising a sequence at least about 80%, at least 85%, at least 90%, at least 92%, at least 95%, at least 97%, at least 99% identical to a sequence set forth in SEQ ID NO: 17 or 51, a FR2 comprising a sequence at least about 80%, at least 85%, at least 90%, at least 92%, at least 95%, at least 97%, at least 99% identical to a sequence set forth in SEQ ID NO: 18 or 52, a FR3 comprising a sequence at least about 80%, at least 85%, at least 90%, at least 92%, at least 95%, at least 97%, at least 99% identical to a sequence set forth in SEQ ID NO: 19 or 53, and a FR4 comprising a sequence at least about 80%, at least 85%, at least 90%, at least 92%, at least 95%, at least 97%, at least 99% identical to a sequence set forth in SEQ ID NO: 20 or 54;
(iii) a VH comprising a FR1 comprising a sequence set forth in SEQ ID NO: 21 or 55, a FR2 comprising a sequence set forth between in SEQ ID NO: 22 or 56, a FR3 comprising a sequence set forth in SEQ ID NO: 23 or 57, and a FR4 comprising a sequence set forth in SEQ ID NO: 24 or 58;
(iv) a VL comprising a FR1 comprising a sequence set forth in SEQ ID NO: 17 or 51, a FR2 comprising a sequence set forth between in SEQ ID NO: 18 or 52, a FR3 comprising a sequence set forth in SEQ ID NO: 19 or 53, and a FR4 comprising a sequence set forth in SEQ ID NO: 20 or 54; or
(v) a VH comprising a FR1 comprising a sequence set forth in SEQ ID NO: 21 or 55, a FR2 comprising a sequence set forth between in SEQ ID NO: 22 or 56, a FR3 comprising a sequence set forth in SEQ ID NO: 23 or 57, and a FR4 comprising a sequence set forth in SEQ ID NO: 24 or 58; and a VL comprising a FR1 comprising a sequence set forth in SEQ ID NO: 17 or 51, a FR2 comprising a sequence set forth between in SEQ ID NO: 18 or 52, a FR3 comprising a sequence set forth in SEQ ID NO: 19 or 53, and a FR4 comprising a sequence set forth in SEQ ID NO: 20 or 54.

32. An antigen binding site according to claim 31, wherein the antigen binding site is in the form of:
(i) a single chain Fv fragment (scFv);
(ii) a dimeric scFv (di-scFv);
(iii) one of (i) or (ii) linked to a constant region of an antibody, Fc or a heavy chain constant domain (CH) 2 and/or CH3; or
(iv) one of (i) or (ii) linked to a protein that binds to tau.

33. An antigen binding site according to claim 31, wherein the antigen binding site is in the form of:
(i) a diabody;
(ii) a triabody;
(iii) a tetrabody;
(iv) a Fab;
(v) a F(ab′)2;
(vi) a Fv;
(vii) one of (i) to (vi) linked to a constant region of an antibody, Fc or a heavy chain constant domain (CH) 2 and/or CH3; or
(viii) one of (i) to (vi) linked to a protein that binds to tau.

34. A fusion protein comprising an antigen binding site according to any one of claims 30 to 33.

35. A nucleic acid encoding an antigen binding site according to any one of claims 30 to 33.

36. A vector comprising a nucleic acid according to claim 35.

37. A cell comprising a vector according to claim 36 or a nucleic acid according to claim 35.

38. A pharmaceutical composition comprising an antigen binding site according to any one of claims 30 to 33 and a pharmaceutically acceptable carrier, diluent or excipient.

39. A method according to any one of claims 1 to 29, wherein the antigen binding site is according to any one of claims 30 to 33.

40. A method for treating, delaying, inhibiting or preventing the progression of a tauopathy comprising administering an antigen binding site according to any one of claims 30 to 33 to a subject in need thereof, thereby treating, delaying, inhibiting or preventing the progression of a tauopathy in a subject in need thereof.

41. Use of an antigen binding site according to any one of claims 30 to 33 in the preparation of a medicament for treating, inhibiting, delaying or reducing the progression of a tauopathy in a subject in need thereof.

42. The antigen binding site of any one of claims 30 to 33, wherein the binding site is chimeric, humanized, human, synhumanized, primatized, de-immunized or a composite antigen binding site.

43. The method according to any one of claims 1 to 29, wherein the acoustic energy is scanning ultrasound (SUS).


SEQUENCE LISTING (66 sequences)

SEQ ID NO: 1 - protein, 12 aa, Homo sapiens
QSLLYSSNQKNY

SEQ ID NO: 2 - protein, 3 aa, Homo sapiens
WAS

SEQ ID NO: 3 - protein, 9 aa, Homo sapiens
QQYYGYPLT

SEQ ID NO: 4 - protein, 8 aa, Homo sapiens
GFSLTSYG

SEQ ID NO: 5 - protein, 6 aa, Homo sapiens
IWRGGS

SEQ ID NO: 6 - protein, 12 aa, Homo sapiens
AKNTNHRYDGYY

SEQ ID NO: 7 - protein, 113 aa, Homo sapiens
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYGYPLTFGAGTKLELK

SEQ ID NO: 8 - protein, 121 aa, Homo sapiens
QVQLKQSGPGLVQPSQSLSITCTVSGFSLTSYGVHWVRQSPGKGLEWLGVIWRGGSTDYNAAFMSRLSITKDNSKSQVFFKMNSLQADDTAIYYCAKNTNHRYDGYYAMDYWGQGTSVTVS

SEQ ID NO: 9 - DNA, 36 bp, Homo sapiens
caatccctgctctactcttcaaatcagaaaaactat

SEQ ID NO: 10 - DNA, 9 bp, Homo sapiens
tgggcaagt

SEQ ID NO: 11 - DNA, 27 bp, Homo sapiens
cagcaatattacgggtaccccttgaca

SEQ ID NO: 12 - DNA, 24 bp, Homo sapiens
gggttcagtctcacttcctatggt

SEQ ID NO: 13 - DNA, 18 bp, Homo sapiens
atatggcgaggggggtcc

SEQ ID NO: 14 - DNA, 36 bp, Homo sapiens
gcaaagaatacaaaccacaggtatgatggatactac

SEQ ID NO: 15 - DNA, 339 bp, Homo sapiens
gatatagttatgtctcaaagtccttcaagcctcgcagttagtgttggtgaaaaagtaacaatgagctgcaaatcatctcaatccctgctctactcttcaaatcagaaaaactatttggcttggtatcaacagaagcccggacaaagtccaaagttgctcatatactgggcaagtactagagagtccggtgtccccgatagatttacaggcagtggctcaggaaccgacttcactttgaccataagttctgtgaaggcagaggatttggcagtttattattgtcagcaatattacgggtaccccttgacatttggagccgggactaaacttgagctgaag

SEQ ID NO: 16 - DNA, 363 bp, Homo sapiens
caagtccagttgaagcagagcggccccggtctcgtccaacctagccaaagcttgtccataacttgtacagtatcagggttcagtctcacttcctatggtgtgcactgggtccgccagagtcctggcaagggcctcgaatggctcggagtaatatggcgaggggggtccactgactataatgccgcttttatgagtaggctctctataactaaggacaattctaagagtcaggtcttcttcaaaatgaactcccttcaggcagacgataccgctatctattactgtgcaaagaatacaaaccacaggtatgatggatactacgctatggattattggggtcaaggcaccagcgtcactgtctcc

SEQ ID NO: 17 - protein, 26 aa, Homo sapiens
DIVMSQSPSSLAVSVGEKVTMSCKSS

SEQ ID NO: 18 - protein, 17 aa, Homo sapiens
LAWYQQKPGQSPKLLIY

SEQ ID NO: 19 - protein, 36 aa, Homo sapiens
TRESGVPDRFTGSGSGTDFTLTISSVKAEDLAVYYC

SEQ ID NO: 20 - protein, 10 aa, Homo sapiens
FGAGTKLELK

SEQ ID NO: 21 - protein, 25 aa, Homo sapiens
QVQLKQSGPGLVQPSQSLSITCTVS

SEQ ID NO: 22 - protein, 17 aa, Homo sapiens
VHWVRQSPGKGLEWLGV

SEQ ID NO: 23 - protein, 39 aa, Homo sapiens
TDYNAAFMSRLSITKDNSKSQVFFKMNSLQADDTAIYYC

SEQ ID NO: 24 - protein, 14 aa, Homo sapiens
AMDYWGQGTSVTVS

SEQ ID NO: 25 - DNA, 78 bp, Homo sapiens
gatatagttatgtctcaaagtccttcaagcctcgcagttagtgttggtgaaaaagtaacaatgagctgcaaatcatct

SEQ ID NO: 26 - DNA, 51 bp, Homo sapiens
ttggcttggtatcaacagaagcccggacaaagtccaaagttgctcatatac

SEQ ID NO: 27 - DNA, 108 bp, Homo sapiens
actagagagtccggtgtccccgatagatttacaggcagtggctcaggaaccgacttcactttgaccataagttctgtgaaggcagaggatttggcagtttattattgt

SEQ ID NO: 28 - DNA, 30 bp, Homo sapiens
tttggagccgggactaaacttgagctgaag

SEQ ID NO: 29 - DNA, 75 bp, Homo sapiens
caagtccagttgaagcagagcggccccggtctcgtccaacctagccaaagcttgtccataacttgtacagtatca

SEQ ID NO: 30 - DNA, 51 bp, Homo sapiens
gtgcactgggtccgccagagtcctggcaagggcctcgaatggctcggagta

SEQ ID NO: 31 - DNA, 117 bp, Homo sapiens
actgactataatgccgcttttatgagtaggctctctataactaaggacaattctaagagtcaggtcttcttcaaaatgaactcccttcaggcagacgataccgctatctattactgt

SEQ ID NO: 32 - DNA, 42 bp, Homo sapiens
gctatggattattggggtcaaggcaccagcgtcactgtctcc

SEQ ID NO: 33 - protein, 440 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQG

SEQ ID NO: 34 - protein, 14 aa, Homo sapiens
TEIPEGITAEEAGI

SEQ ID NO: 35 - protein, 17 aa, Homo sapiens
KSSQSLLYSSNQKNYLA

SEQ ID NO: 36 - protein, 7 aa, Homo sapiens
WASTRES

SEQ ID NO: 37 - protein, 9 aa, Homo sapiens
QQYYGYPLT

SEQ ID NO: 38 - protein, 7 aa, Homo sapiens
GFSLTSY

SEQ ID NO: 39 - protein, 7 aa, Homo sapiens
VIWRGGS

SEQ ID NO: 40 - protein, 14 aa, Homo sapiens
NTNHRYDGYYAMDY

SEQ ID NO: 41 - protein, 113 aa, Homo sapiens; identical to SEQ ID NO: 7

SEQ ID NO: 42 - protein, 121 aa, Homo sapiens; identical to SEQ ID NO: 8

SEQ ID NO: 43 - DNA, 51 bp, Homo sapiens
aaatcatctcaatccctgctctactcttcaaatcagaaaaactatttggct

SEQ ID NO: 44 - DNA, 21 bp, Homo sapiens
tgggcaagtactagagagtcc

SEQ ID NO: 45 - DNA, 27 bp, Homo sapiens
cagcaatattacgggtaccccttgaca

SEQ ID NO: 46 - DNA, 21 bp, Homo sapiens
gggttcagtctcacttcctat

SEQ ID NO: 47 - DNA, 21 bp, Homo sapiens
gtaatatggcgaggggggtcc

SEQ ID NO: 48 - DNA, 42 bp, Homo sapiens
aatacaaaccacaggtatgatggatactacgctatggattat

SEQ ID NO: 49 - DNA, 339 bp, Homo sapiens; identical to SEQ ID NO: 15

SEQ ID NO: 50 - DNA, 363 bp, Homo sapiens; identical to SEQ ID NO: 16

SEQ ID NO: 51 - protein, 23 aa, Homo sapiens
DIVMSQSPSSLAVSVGEKVTMSC

SEQ ID NO: 52 - protein, 15 aa, Homo sapiens
WYQQKPGQSPKLLIY

SEQ ID NO: 53 - protein, 32 aa, Homo sapiens
GVPDRFTGSGSGTDFTLTISSVKAEDLAVYYC

SEQ ID NO: 54 - protein, 10 aa, Homo sapiens
FGAGTKLELK

SEQ ID NO: 55 - protein, 25 aa, Homo sapiens
QVQLKQSGPGLVQPSQSLSITCTVS

SEQ ID NO: 56 - protein, 17 aa, Homo sapiens
GVHWVRQSPGKGLEWLG

SEQ ID NO: 57 - protein, 41 aa, Homo sapiens
TDYNAAFMSRLSITKDNSKSQVFFKMNSLQADDTAIYYCAK

SEQ ID NO: 58 - protein, 10 aa, Homo sapiens
WGQGTSVTVS

SEQ ID NO: 59 - DNA, 69 bp, Homo sapiens
gatatagttatgtctcaaagtccttcaagcctcgcagttagtgttggtgaaaaagtaacaatgagctgc

SEQ ID NO: 60 - DNA, 45 bp, Homo sapiens
tggtatcaacagaagcccggacaaagtccaaagttgctcatatac

SEQ ID NO: 61 - DNA, 96 bp, Homo sapiens
ggtgtccccgatagatttacaggcagtggctcaggaaccgacttcactttgaccataagttctgtgaaggcagaggatttggcagtttattattgt

SEQ ID NO: 62 - DNA, 30 bp, Homo sapiens; identical to SEQ ID NO: 28

SEQ ID NO: 63 - DNA, 75 bp, Homo sapiens; identical to SEQ ID NO: 29

SEQ ID NO: 64 - DNA, 51 bp, Homo sapiens
ggtgtgcactgggtccgccagagtcctggcaagggcctcgaatggctcgga

SEQ ID NO: 65 - DNA, 123 bp, Homo sapiens
actgactataatgccgcttttatgagtaggctctctataactaaggacaattctaagagtcaggtcttcttcaaaatgaactcccttcaggcagacgataccgctatctattactgtgcaaag

SEQ ID NO: 66 - DNA, 30 bp, Homo sapiens
tggggtcaaggcaccagcgtcactgtctcc